SEQUENCE LISTING (2 sequences)

SEQ ID NO: 1 - protein, 44 aa, Artificial Sequence
  Synthetic Peptide
HGEGTFTSDLSKQMEEEAVRLFIEWLKNGGPSSGAPPSKKKKKK

SEQ ID NO: 2 - protein, 39 aa, Artificial Sequence
  Synthetic Peptide
HGEGTFTSDLSKQMEEEAVRLFIEWLKNGGPSSGAPPPS

(19) United States
(12) Patent Application Publication
(10) Pub. No.: US 2013/0085102 A1
(43) Pub. Date: Apr. 4, 2013

(54) PREVENTION OF HYPOGLYCEMIA IN DIABETES MELLITUS TYPE 2 PATIENTS

(71) Applicants: Louise SILVESTRE, Paris (FR); Gabor BOKA, Paris (FR); Patrick MIOSSEC, Paris (FR)

(72) Inventors: Louise SILVESTRE, Paris (FR); Gabor BOKA, Paris (FR); Patrick MIOSSEC, Paris (FR)

(73) Assignee: SANOFI-AVENTIS DEUTSCHLAND GMBH, Frankfurt (DE)

(21) Appl. No.: 13/363,956

(22) Filed: Feb. 1, 2012

(30) Foreign Application Priority Data
Feb. 2, 2011 (EP) 11153106.7

(51) Int. Cl. A61K 38/26; A61K 31/155

(52) U.S. Cl. CPC A61K 38/26; A61K 31/155
USPC 514/7.2

(57) ABSTRACT

A method for the prevention of hypoglycaemia in diabetes mellitus type 2 comprising administering
(a) desPro³⁶Exendin-4(1-39)-Lys₆-NH₂ or/and a pharmaceutically acceptable salt thereof, and
(b) metformin or/and a pharmaceutically acceptable salt thereof,
to a subject in need thereof.

Subject of the present invention is a method for treatment of diabetes mellitus type 2 with AVE0010 (lixisenatide) as add-on therapy to administration of metformin.

Metformin is a biguanide hypoglycemic agent used in the treatment of Type 2 diabetes mellitus not responding to dietary modification. Metformin improves glycemic control by improving insulin sensitivity. Metformin is usually administered orally. However, control diabetes mellitus type 2 in obese patients by metformin may be insufficient. Thus, in these patients, additional measures for controlling diabetes mellitus type 2 may be required.

Hypoglycaemia is the critical limiting factor in the glycaemic management of diabetes in both the short and long term. Despite steady improvements in the glycaemic management of diabetes, population-based data indicate that hypoglycaemia continues to be a major problem for people with both type 1 and type 2 diabetes (American diabetes association, workgroup on hypoglycemia: Defining and Reporting Hypoglycemia in Diabetes. Diabetes Care 28(5), 2005, 1245-1249).

A first aspect of the present invention is a method for the treatment of diabetes mellitus type 2 comprising administering
(a) desPro³⁶Exendin-4(1-39)-Lys₆-NH₂ or/and a pharmaceutically acceptable salt thereof, and
(b) metformin or/and a pharmaceutically acceptable salt thereof,
   to a subject in need thereof.

In particular, the method is a method for the prevention of hypoglycaemia in a diabetes mellitus type 2 patient. More particular, the method is a method for the prevention of symptomatic hypoglycaemia or severe symptomatic hypoglycaemia in a diabetes mellitus type 2 patient.

More particular, the method of the present invention is a method for the prevention of hypoglycaemia in a diabetes type 2 patient having an increased risk of hypoglycaemia, in particular a diabetes type 2 patient having experienced at least one hypoglycaemic event. The hypoglycaemic event can be a symptomatic hypoglycaemic event or a severe symptomatic hypoglycaemic event.

In the present invention, hypoglycaemia is a condition wherein a diabetes mellitus type 2 patient experiences a plasma glucose concentration of below 60 mg/dL (or below 3.3 mmol/L), below 50 mg/dL, below 40 mg/dL, or below 36 mg/dL.

By the method of the present invention, hypoglycaemia can be reduced to below 12%, below 11%, below 10%, below 9%, below 8%, below 7%, below 6% or below 5% of diabetes type 2 patients receiving the combination of lixisenatide and metformin, as described herein.

In the present invention, “symptomatic hypoglycaemia” or “symptomatic hypoglycaemic event” is a condition associated with a clinical symptom that results from the hypoglycaemia, wherein the plasma glucose concentration is below 60 mg/dL (or below 3.3 mmol/L), below 50 mg/dL, or below 40 mg/dL. A clinical symptoms can be, for example, sweating, palpitations, hunger, restlessness, anxiety, fatigue, irritability, headache, loss of concentration, somnolence, psychiatric disorders, visual disorders, transient sensory defects, transient motor defects, confusion, convulsions, and coma. In the method of the present invention, one or more clinical symptoms of symptomatic hypoglycaemia, as indicated herein, can be selected. Symptomatic hypoglycaemia may be associated with prompt recovery after oral carbohydrate administration.

In the present invention, “severe symptomatic hypoglycaemia” or “severe symptomatic hypoglycaemic event” is a condition with a clinical symptom, as indicated herein, that results from hypoglycaemia, wherein the plasma glucose concentration is below 36 mg/dL (or below 2.0 mmol/L). Severe symptomatic hypoglycaemia can be associated with acute neurological impairment resulting from the hypoglycaemic event. In a severe symptomatic hypoglycaemia, the patient may require the assistance of another person, if, for example, the patient could not treat or help him/herself due to the acute neurological impairment. The definition of severe symptomatic hypoglycaemia may include all episodes in which neurological impairment is severe enough to prevent self-treatment and which were thus thought to place patients at risk for injury to themselves or others. The acute neurological impairment may be at least one selected from somnolence, psychiatric disorders, visual disorders, transient sensory defects, transient motor defects, confusion, convulsions, and coma.

Severe symptomatic hypoglycaemia may be associated with prompt recovery after oral carbohydrate, intravenous glucose, or/and glucagon administration.

Normoglycaemia may relate to a blood plasma concentration of glucose of from 60 mg/dL to 140 mg/dL (corresponding to 3.3 mmol/L to 7.8 mmol/L).

It has surprisingly been found in a clinical trial that during treatment of diabetes mellitus type 2 patients with lixisenatide combined with metformin, only 5% of patients had symptomatic hypoglycaemic events, whereas in a comparative trial, 14.6% of diabetes mellitus type 2 patients treated with a combination of exenatide and metformin reported symptomatic hypoglycaemia during the same period. This results indicate that the combination of lixisenatide and metformin can be used for the prevention of hypoglycaemia.

The combination of lixisenatide and metformin, as described herein, can also be used for the reduction or/and prevention of side effects of anti-diabetic treatment in diabetes mellitus type 2 patients.

In the present invention, side effects of the combination of lixisenatide and metformin are investigated in a clinical trail of treatment of diabetes mellitus type 2 patients with lixisenatide combined with metformin (Example 2). In this trial, side effects are described by treatment emergent adverse events (TEAEs).

The side effect may be a gastrointestinal motility and defaecation condition, for example diarrhoea, non-infective diarrhoea, a gastrointestinal atonic and hypomotility disorder NEC, constipation, gastrooesophageal reflux disease. The side effect may also by a gastrointestinal sign and symptom, for example a dyspeptic sign and symptom, dyspepsia, flatulence, bloating, distension, abdominal distension, gastrointestinal and abdominal pain (for example, excluding oral and throat pain), abdominal pain, pain of the upper abdomen, abdominal discomfort, a nausea or/and vomiting symptom, nausea or vomiting. In particular, the side effect is nausea or vomiting. More particular, the side effect is nausea.

It has surprisingly been found that in the clincal trial, side effects were reduced, for example nausea (see, for example, Table 29 of Example 2), compared with a comparative trial of treatment of diabetes mellitus type 2 patients with a combination of exenatide and metformin.

The side effect may also be pancreatitis. During the on-treatment period of the clinical trial, 5 (1.6%) lixisenatide-treated patients and 9 (2.8%) exenatide-treated patients reported events of changes in pancreatic enzymes or lipase or amylase for “suspected pancreatitis” (Tables 23 and 24 of Example 2). However, no case of acute pancreatitis was observed.

The side effect may also be an increased blood calcitonin concentration. In the clinical trial, eight patients (4 [1.3%] in each group) reported a calcitonin value ≧20 ng/L (Table 25). No value ≧50 ng/L was reported. Five (1.8%) patients in the lixisenatide group and 8 (3.0%) patients in the exenatide group had a value of calcitonin ≧20 ng/L during the on treatment period (Table 26).

These results indicate that the combination of lixisenatide and metformin can be used for the reduction or/and prevention of side effects of anti-diabetic treatment in diabetes mellitus type 2 patients. In particular, these results indicate that the combination of lixisenatide and metformin can be used for the reduction or/and prevention of nausea, pancreatitis or/and increased blood calcitonin concentration.

The compounds of (a) and (b) may be administered to a subject in need thereof, in an amount sufficient to induce a therapeutic effect.

The compound desPro³⁶Exendin-4(1-39)-Lys₆-NH₂ (AVE0010, lixisenatide) is a derivative of Exendin-4. AVE0010 is disclosed as SEQ ID NO:93 in WO 01/04156:

SEQ ID NO: 1
AVE0010 (44 AS)
H-G-E-G-T-F-T-S-D-L-S-K-Q-M-E-E-E-A-V-R-L-F-1-E-W-
L-K-N-G-G-P-S-S-G-A-P-P-S-K-K-K-K-K-K-NH₂
SEQ ID NO: 2
Exendin-4 (39 AS)
H-G-E-G-T-F-T-S-D-L-S-K-Q-M-E-E-E-A-V-R-L-F-I-E-W-
L-K-N-G-G-P-S-S-G-A-P-P-P-S-NH₂

Exendins are a group of peptides which can lower blood glucose concentration. The Exendin analogue AVE0010 is characterised by C-terminal truncation of the native Exendin-4 sequence. AVE0010 comprises six C-terminal lysine residues not present in Exendin-4.

In the context of the present invention, AVE0010 includes pharmaceutically acceptable salts thereof. The person skilled in the art knows pharmaceutically acceptable salts of AVE0010. A preferred pharmaceutically acceptable salt of AVE0010 employed in the present invention is acetate.

AVE0010 (desPro³⁶Exendin-4(1-39)-Lys₆-NH₂) or/and a pharmaceutically acceptable salt thereof may be administered by subcutaneous injection. Suitable injection devices, for instance the so-called “pens” comprising a cartridge comprising the active ingredient, and an injection needle, are known. AVE0010 or/and a pharmaceutically acceptable salt thereof may be administered in a suitable amount, for instance in an amount in the range of 10 to 15 μg per dose or 15 to 20 μg per dose once a day (progressive titration from 10 to 15 and to 20 μg/day. 20 μg is the effective maintenance dose).

In the present invention, AVE0010 or/and a pharmaceutically acceptable salt thereof may be administered in a daily dose in the range of 10 to 15 μg or in the range of 15 to 20 μg once a day (progressive titration from 10 to 15 and to 20 μg/day. 20 μg is the effective maintenance dose). AVE0010 or/and a pharmaceutically acceptable salt thereof may be administered by one injection per day.

In the present invention, a liquid composition comprising desPro³⁶Exendin-4(1-39)-Lys₆-NH₂ or/and a pharmaceutically acceptable salt thereof may be employed. The skilled person knows liquid compositions of AVE0010 suitable for parenteral administration. A liquid composition of the present invention may have an acidic or a physiologic pH. An acidic pH preferably is in the range of pH 1-6.8, pH 3.5-6.8, or pH 3.5-5. A physiologic pH preferably is in the range of pH 2.5-8.5, pH 4.0 to 8.5, or pH 6.0 to 8.5. The pH may be adjusted by a pharmaceutically acceptable diluted acid (typically HCl) or pharmaceutically acceptable diluted base (typically NaOH).

The preferred pH is in the range of pH 3.5 to 5.0.

The liquid composition may contain a buffer, such as a phosphate, a citrate, an acetate.

Preferably, it can contain an acetate buffer, in quantities up to 5 μg/mL, up to 4 μg/mL or up to 2 μg/mL.

The liquid composition of the present invention may comprise a suitable preservative. A suitable preservative may be selected from phenol, m-cresol, benzyl alcohol and p-hydroxybenzoic acid ester. A preferred preservative is m-cresol.

The liquid composition of the present invention may comprise a tonicity agent. A suitable tonicity agent may be selected from glycerol, lactose, sorbitol, mannitol, glucose, NaCl, calcium or magnesium containing compounds such as CaCl₂. The concentration of glycerol, lactose, sorbitol, mannitol and glucose may be in the range of 100-250 mM. The concentration of NaCl may be up to 150 mM. A preferred tonicity agent is glycerol.

In addition, the liquid composition may contain L-methionin from 0.5 μg/mL to 20 μg/mL, preferably from 1 μg/mL to 5 μg/mL. Preferably, it contains L-methionin.

Metformin is the international non proprietary name of 1,1-dimethylbiguanide (CAS Number 657-24-9). In the present invention, the term “metformin” includes any pharmaceutically acceptable salt thereof.

In the present invention, metformin may be administered orally. The skilled person knows formulations of metformin suitable for treatment of diabetes type 2 by oral administration. Metformin may be administered in a dose of at least 1.0 g/day or at least 1.5 g/day. For oral administration, metformin may be formulated in a solid dosage form, such as a tablet or pill.

In the present invention, desPro³⁶Exendin-4(1-39)-Lys₆-NH₂ or/and a pharmaceutically acceptable salt is administered in an add-on therapy to administration of metformin.

In the present invention, the terms “add-on”, “add-on treatment” and “add-on therapy” relate to treatment of diabetes mellitus type 2 with metformin and AVE0010. Metformin and AVE0010 may be administered within a time interval of 24 h. Metformin and AVE0010 each may be administered in a once-a-day-dosage. Metformin and AVE0010 may be administered by different administration routes. Metformin may be administered orally, and AVE0010 may be administered subcutaneously.

The subject to be treated by the method of the present invention suffering from diabetes type 2 may be an obese subject. In the present invention, an obese subject may have a body mass index of at least 30.

The subject to be treated by the method of the present invention may have a HbA1c value in the range of 7% to 10%.

The subject to be treated by the method of the present invention may have a HbA1c value of at least 8%, In particular, the subject to be treated by the method of the present invention may have a HbA1c value in the range of 8% to 10%.

The subject to be treated by the method of the present invention may have a HbA1c value of below 8%, In particular, the subject to be treated by the method of the present invention may have a HbA1c value in the range of 7% to 8%.

The subject to be treated by the method of the present invention may be an adult subject. The subject may have an age in the range of 18 to 50 years.

The method of the present invention preferably is a method of treatment of a subject suffering from diabetes type 2, wherein diabetes type 2 is not adequately controlled by treatment with metformin alone, for instance with a dose of at least 1.0 g/day metformin or at least 1.5 g/day metformin for 3 months. In the present invention, a subject the diabetes type 2 of which is not adequately controlled may have a HbA1c value in the range of 7% to 10%.

Another aspect of the present invention is a pharmaceutical combination comprising
(a) desPro³⁶Exendin-4(1-39)-Lys₆-NH₂ or/and a pharmaceutically acceptable salt thereof, and
(b) metformin or/and a pharmaceutically acceptable salt thereof.

Preferably, the combination of the present invention is for use in the treatment of diabetes mellitus type 2.

Preferably, the combination of the present invention is for use in the prevention of hypoglycaemia, as described herein, in diabetes mellitus type 2 patients.

More preferably the combination of the present invention is for use in the prevention of hypoglycaemia in a diabetes type 2 patient having an increased risk of hypoglycaemia, in particular a diabetes type 2 patient having experienced at least one hypoglycaemic event. The hypoglycaemic event can be a symptomatic hypoglycaemic event or a severe symptomatic hypoglycaemic event.

Preferably, the combination of the present invention is for use in the prevention of side effects of anti-diabetic treatment, as described herein, in diabetes mellitus type 2 patients. In particular, the side effect is nausea, pancreatitis or/and increased blood calcitonin concentration.

The combination of the present invention may be administered as described herein in the context of the method of the present invention. The compounds (a) and (b) of the combination of the present invention may be formulated as described herein in the context of the method of the present invention.

Yet another aspect of the present invention is the use of a combination comprising
(a) desPro³⁶Exendin-4(1-39)-Lys₆-NH₂ or/and a pharmaceutically acceptable salt thereof, and
(b) metformin or/and a pharmaceutically acceptable salt thereof,
   for the production of a medicament for the treatment of diabetes mellitus type 2.

The medicament comprises desPro³⁶Exendin-4(1-39)-Lys₆-NH₂ and metformin in separate formulations, as described herein.

The combination of the present invention can be used for production of a medicament for the prevention of hypoglycaemia, as described herein, in diabetes mellitus type 2 patients.

The combination of the present invention can be used for production of a medicament for the prevention of side effects of anti-diabetic treatment in diabetes mellitus type 2 patients, as described herein. In particular, the side effect is nausea, pancreatitis or/and increased blood calcitonin concentration.

The invention is further illustrated by the following Examples and Figures.

### LEGENDS OF THE FIGURES

FIG. 1: Study design of Example 2.

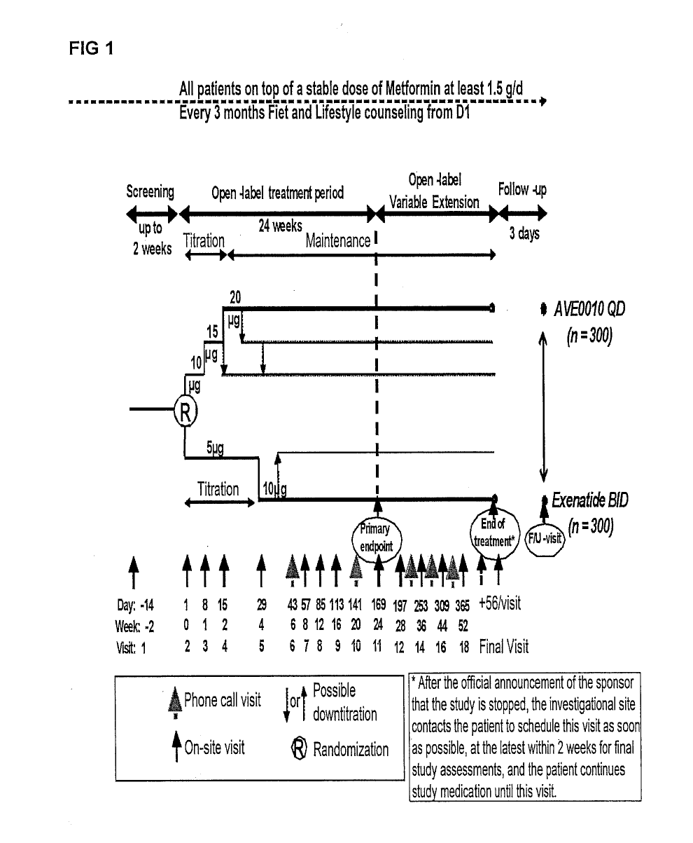

FIG. 2: Kaplan-Meier plot of time to treatment discontinuation due to any reason—randomized population.

FIG. 3: Plot of mean change in HbA1C (%) from baseline by visit and at endpoint mITT population. EOT=last value on-treatment (LOCF). LOCF=Last observation carry forward. Note: The analysis excluded measurements obtained after the introduction of rescue medication and/or after the treatment cessation plus 3 days. For Week 24 (LOCF), the analysis included measurements obtained up to 3 days after the last dose of the investigational product injection on or before Visit 11 (Week 24), or Day 169 if Visit 11 (Week 24) is not available.

FIG. 4: Plot of mean change in fasting plasma glucose (mmol/L) from baseline by visit and at endpoint-mITT population. EOT=last value on-treatment (LOCF). LOCF=Last observation carry forward. Note: The analysis excluded measurements obtained after the introduction of rescue medication and/or after the treatment cessation plus 3 days. For Week 24 (LOCF), the analysis included measurements obtained up to 1 days after the last dose of the investigational product injection on or before Visit 11 (Week 24), or Day 169 if Visit 11 (Week 24) is not available.

FIG. 5: Plot of mean change in body weight (kg) from baseline by visit and at endpoint-mITT population. EOT=last value on-treatment (LOCF). LOCF=Last observation carry forward. Note: The analysis excluded measurements obtained after the introduction of rescue medication and/or after the treatment cessation plus 3 days. For Week 24 (LOCF), the analysis included measurements obtained up to 3 days after the last dose of the investigational product injection on or before Visit 11 (Week 24), or Day 169 if Visit 11 (Week 24) is not available.

### EXAMPLE 1

### 24-Week Study Comparing Lixisenatide (AVE0010) to Sitagliptin as Add-on to Metformin in Obese Type 2 Diabetic Patients Younger than 50

Subject of the example is a randomized, double-blind, double-dummy, 2-arm parallel-group, multicenter, 24-week study comparing the efficacy and safety of lixisenatide (AVE0010) to sitagliptin (CAS Number 486460-32-6) as add-on to metformin in obese type 2 diabetic patients younger than 50 years and not adequately controlled with metformin Sitagliptin is an antidiabetic drug, acting as an inhibitor of dipeptidyl peptidase 4 (DPP4) resulting in enhanced level of Glucagon-Like Peptide 1, thereby reducing blood glucose levels in diabetic patients.

### Study Primary Objectives

The primary objective of this study is to assess the efficacy of lixisenatide on a composite endpoint of glycemic control (HbA1c) and body weight in comparison to sitagliptin as an add-on treatment to metformin over a period of 24 weeks in obese type 2 diabetic patients younger than 50.

### Study Secondary Objectives are Assessment of the Effects of Lixisenatide on:

Absolute changes in HbA1c and body weight
Fasting plasma glucose
Plasma glucose, insulin, C peptide, glucagon and proinsulin during a 2-hour standardized meal test
Insulin resistance assessed by HOMA-IR
Beta cell function assessed by HOMA-beta
To assess lixisenatide safety and tolerability
To assess lixisenatide PK using the population PK approach and to assess anti-lixisenatide antibody development.

### Specific Vulnerable Populations:

Women of child-bearing potential using contraception.

### Inclusion Criteria

Patients (male and female) with type 2 diabetes mellitus, as defined by WHO (21), diagnosed for at least 1 year at the time of screening visit, insufficiently controlled with metformin at a stable dose of at least 1.5 g/day, for at least 3 months prior to the screening visit. Patients with obesity (BMI ≧30 kg/m²) and aged from 18 years to less than 50 years.

### Exclusion Criteria

HbA1c <7.0% or HbA1c >10% at screening
Type 1 diabetes mellitus
Pregnancy or lactation
Women of childbearing potential with no effective contraceptive method
Fasting Plasma Glucose at screening >250 mg/dL (>13.9 mmol/L)
Weight change of more than 5 kg during the 3 months preceding the screening visit
History of unexplained pancreatitis, chronic pancreatitis, pancreatectomy, stomach/gastric surgery, inflammatory bowel disease
History of metabolic acidosis, including diabetic ketoacidosis within 1 year prior to screening
Hemoglobinopathy or hemolytic anemia or receipt of blood or plasma products within 3 months prior to the time of screening
Within the last 6 months prior to screening: history of myocardial infarction, stroke, or heart failure requiring hospitalization
Known history of drug or alcohol abuse within 6 months prior to the time of screening
Any clinically significant abnormality identified on physical examination, laboratory tests, ECG or vital signs at the time of screening that in the judgment of the investigator or any sub investigator would preclude safe completion of the study or constrains efficacy assessment such as major systemic diseases, presence of clinically significant diabetic retinopathy or presence of macular edema likely to require laser treatment within the study period
Uncontrolled or inadequately controlled hypertension at the time of screening with a resting systolic or diastolic blood pressure >180 mmHg or >110 mmHg, respectively
Laboratory findings at the time of screening:
   Amylase and/or lipase >3 times the upper limit of the normal laboratory range
   Total bilirubin: >1.5 times the upper limit of the normal laboratory range (except in case of Gilbert's syndrome)
   Hemoglobin <11 g/dL and/or neutrophils <1,500/mm³ and/or platelets <100,000/mm³
   Positive test for Hepatitis B surface antigen and/or Hepatitis C antibody
   Positive serum pregnancy test in females of childbearing potential
Use of other oral or injectable antidiabetic or hypoglycemic agents than metformin (e.g., sulfonylurea, alpha glucosidase inhibitor, thiazolidinedione, exenatide, DPP-IV inhibitors, insulin etc.) within 3 months prior to the time of screening
Unstable diet or unstable anti-obesity treatment within 3 months prior to the time of screening
Use of systemic glucocorticoids (excluding topical application or inhaled forms) for one week or more within 3 months prior to the time of screening
Use of any investigational drug within 3 months prior to screening
Clinically relevant history of gastrointestinal disease associated with prolonged nausea and vomiting, including, but not limited to gastroparesis and gastroesophageal reflux disease requiring medical treatment, within 6 months prior to the time of screening
Any previous treatment with lixisenatide (e.g. participation in a previous study with lixisenatide)
Allergic reaction to any GLP 1-agonist in the past (e.g. exenatide, liraglutide) or to metacresol
History of a serious hypersensitivity reaction to sitagliptin.
Moderate or severe renal impairment (creatinine clearance inferior to 50 ml/min)

### Duration of Study Period Per Subject

Maximum duration of 27 weeks±7 days (3-week screening+24-week double-blind, double-dummy, active-controlled treatment+3-day follow-up)

### Investigational Products

| | Compound | | |
|---|---|---|---|
| INN | code | Pharmaceutical form | Route of administration |
| Lixisenatide | AVE0010 | injection | subcutaneous |
| Sitagliptin | | capsules | capsules |

### Study Arms Number of Arms: 2

| Arm Label | Arm description | Arm type |
|---|---|---|
| Lixisenatide | Injection of lixisenatide once a day in | Experimental |
| | the morning within 1 hour prior to | |
| | breakfast (first 2 weeks of double-blind | |
| | period: titration 10 to 15 μg, then 15 to | |
| | 20 μg) and one capsule of sitagliptin | |
| | placebo intake in the morning or without | |
| | food. On top of metformin background | |
| | therapy. | |
| Sitagliptin | One capsule of sitagliptin intake in the | Active |
| | morning with or without food and | Calibrator/ |
| | lixisenatide matched placebo injection | Comparator |
| | once a day in the morning within hour | |
| | prior to breakfast. On top of metformin | |
| | background therapy. | |

### Endpoints

| | Time frame |
|---|---|
| | for evaluation |
| Primary Endpoint(s): | |
| Percentage of patients with HbA1c values <7% AND a | 24 weeks |
| weight loss of at least 5% of baseline body weight | |
| Secondary Endpoint(s): | |
| Absolute change in HbA1c values | 24 weeks |
| Percentage of patients with HbA1c values ≦6.5% | 24 weeks |
| Absolute change in body weight | 24 weeks |
| Change in fasting plasma glucose | 24 weeks |
| Change in plasma glucose and in β-cell function during a | 24 weeks |
| test meal | |
| Change in insulin resistance assessed by HOMA-IR | 24 weeks |
| Change in β-cell function assessed by HOMA-β | 24 weeks |
| Percentage of patients requiring rescue therapy during | 24 weeks |
| the double-blind period | |

### EXAMPLE 2

A randomized, open-label, active-controlled, 2-arm parallel-group, multicenter 24 week study followed by an extension assessing the efficacy and safety of AVE0010 versus exenatide on top of metformin in patients with type 2 diabetes not adequately controlled with metformin

### Summary

A randomized, open-label, active-controlled, 2-arm, parallel-group, multicenter, multinational study assessing the efficacy and safety of lixisenatide in comparison to exenatide as an add-on treatment to metformin in patients with type 2 diabetes was performed. The approximate minimum study duration per patient was 78 weeks (up to 2 weeks screening+24-week main treatment+variable extension+3 days follow-up). The study was conducted in 122 centers in 18 countries. The primary objective of the study was to assess the efficacy of lixisenatide on glycemic control in comparison to exenatide in terms of HbA_{1c} reduction (absolute change) over a period of 24 weeks.

A total of 634 patients were randomized to one of the two treatment groups (318 in the lixisenatide group and 316 in the exenatide group). All randomized patients were exposed to the study treatment. Demographics and baseline characteristics were generally similar across the treatment groups. Eighteen patients (7 patients in lixisenatide and 11 patients in exenatide) were excluded from the mITT population for efficacy analyses due to lack of post-baseline efficacy data. During the overall study treatment period, 198 (31.2%) patients prematurely discontinued the study treatment. The percentages of patients who discontinued the treatment were similar between treatment groups (32.1% for lixisenatide and 30.4% for exenatide). The main reason for treatment discontinuation was “adverse events” (14.2% in each group) followed by “other reasons” (9.1% for lixisenatide and 9.8% for exenatide), “lack of efficacy” (6.0% for lixisenatide and 1.9% for exenatide) and “poor compliance to protocol” (2.2% for lixisenatide and 4.1% for exenatide).

The least squared (LS) mean changes from baseline to Week 24 in HbA1c were −0.79% for the lixisenatide group and −0.96% for the exenatide group (LS mean difference vs. exenatide=0.17%). The non-inferiority of lixisenatide compared to exenatide was demonstrated, as the upper bound of the two-sided 95% CI of the LS mean difference was less than the predefined non-inferiority margin of 0.4%. Superiority of lixisenatide over exenatide was not demonstrated.

Lixisenatide was well tolerated. Overall incidence of treatment emergent adverse events (TEAEs) was comparable between the two treatment groups. Six patients (3 patients in each treatment) had SAEs on-treatment leading to death. Forty-eight serious TEAEs occurred during the on-treatment period of the whole study with a similar incidence rate in each treatment group (8.2% for lixisenatide and 7.0% for exenatide). The most commonly reported TEAE was nausea (28.6% for lixisenatide-treated patients, 37.7% exenatide treated patients). Sixteen (5.0%) lixisenatide-treated patients had symptomatic hypoglycemia events as defined in the protocol during the on-treatment period whereas 46 (14.6%) exenatide-treated patients reported symptomatic hypoglycemia during the same period. None of symptomatic hypoglycemia events were severe. A total of 9 patients (6 [1.9%] lixisenatide-treated patients and 3 [0.9%] exenatide-treated patients) reported events adjudicated as an allergic reaction by the Allergic Reaction Assessment Committee (ARAC) but none of them were adjudicated as possibly related to the investigational product.

### 1. Objectives

Primary Objective

The primary objective of this study was to assess the efficacy of lixisenatide on glycemic control in comparison to exenatide as an add-on treatment to metformin in terms of HbA_{1c} reduction over a period of 24 weeks in patients with type 2 diabetes.

Secondary Objective(s)
(c) To assess the efficacy of lixisenatide, in comparison to exenatide, on:
   Percentage of patients reaching HbA_{1c}<7% or HbA_{1c}≦6.5%,
   FPG,
   Body weight,
(d) To assess lixisenatide safety and tolerability,
(e) To assess the impact of gastrointestinal tolerability on quality of life (Patient Assessment of upper GastroIntestinal disorders-Quality Of Live, PAGI-QOL).

Trial Design

This was a randomized, open-label, active-controlled, 2-arm, parallel-group, multicenter, multinational study planned in 300 lixisenatide treated and 300 exenatide treated patients.

The patients were stratified by screening values of HbA_{1c} (<8.0%, ≧8.0%) and Body Mass Index (BMI <30, ≧30 kg/m²).

Per the protocol amendment 4 (dated on 18 Jan. 2010), the approximate minimum study duration per patient was 78 weeks (up to 2 weeks screening+24 weeks main open-label treatment+variable extension+3 days follow-up). Patients who completed the 24-week main open-label period underwent a variable open label extension period, which ended for all patients approximately at the scheduled date of week 76 visit (V24) for the last randomized patient.
Patients who prematurely discontinued the study treatment were continued in the study up to the scheduled date of study completion. They were followed up according to the study procedures as specified in the protocol amendment (except for 3-day safety post-treatment follow-up and PAGI-QOL questionnaire).

Primary and Key Secondary Endpoints

Primary Endpoint

The primary efficacy variable was the absolute change in HbA_{1c} from baseline to week 24, which was defined as: HbA_{1c} at week 24-HbA_{1c} at baseline.

If a patient discontinued the treatment prematurely or received rescue therapy during the main 24-week open-label treatment period or did not have HbA_{1c} value at week 24 visit, the last post-baseline on-treatment HbA_{1c} measurement during the main 24-week on-treatment period was used as HbA_{1c} value at week 24 (Last Observation Carry Forward [LOCF] procedure).

Secondary Endpoints

Efficacy Endpoints

The same procedure for handling missing assessment/early discontinuation was applied as for the primary endpoint.

The secondary efficacy variables were:
Percentage of patients with HbA_{1c}<7% at week 24,
Percentage of patients with HbA_{1c}≦6.5% at week 24,
Change in fasting plasma glucose (mmol/L) (by central laboratory) from baseline to week 24,
Change in body weight (kg) from baseline to week 24,
Percentage of patients requiring rescue therapy during the main 24-week treatment period.
Percentage of patients with ≧5% weight loss (kg) from baseline at week 24.

All secondary endpoints at the end of treatment were to be evaluated by descriptive statistics only (presented in CSR)

Safety Endpoints

The safety analysis was based on the reported TEAEs and other safety information including symptomatic hypoglycemia and severe symptomatic hypoglycemia, local tolerability at injection site, allergic events (as adjudicated by ARAC), suspected pancreatitis, increased calcitonin, vital signs, 12-lead ECG and laboratory tests.

Major cardiovascular events were also collected and adjudicated by a Cardiovascular Adjudication Committee (CAC). The adjudicated and confirmed events by CAC from this study and other lixisenatide phase 2-3 studies will be pooled for analyses and summarized in a separate report based on the statistical analysis plan for the overall cardiovascular assessment of lixisenatide. The KRM/CSR will not present the summary of the adjudicated and confirmed CV events from this study.

Health Related Quality-Of-Life Variables (PAGI-QOL Questionnaire)

The same procedure for handling missing assessment/early discontinuation was applied as for the primary endpoint. The consequence of the gastrointestinal tolerability on health related quality of life was evaluated by the PAGI-QOL questionnaire, which consisted of 30 questions and covered five dimensions including daily activities, clothing, diet and food habits, relationship and psychological well-being and distress. The total score was calculated by taking the mean of the five dimension scores (subscale scores) and ranged from 0 to 5 with lower scores indicating better quality of life. Change in PAGI-QOL total score from baseline to week 24 is analyzed.

Sample Size Calculation Assumptions

The sample size/power calculations were performed based on the primary variable, change from baseline to week 24 in HbA_{1c}.

A sample size of 600 (300 patients in each group) ensured that the upper confidence limit of the two-side 95% confidence interval for the adjusted mean difference between lixisenatide and exenatide would not exceed 0.4% HbA_{1c} with 96% power assuming that the standard deviation was 1.3 and the true difference between lixisenatide and exenatide was zero in HbA_{1c}. Standard deviation was estimated in a conservative manner from previously conducted diabetes studies (based on published data of similarly designed study and on internal data, not published), taking into account early dropout.

Statistical Methods

Analysis Populations

The modified intent-to-treat (mITT) population consisted of all randomized patients who received at least one dose of open-label investigational product (IP), and had both a baseline assessment and at least one post-baseline assessment of efficacy variables.

The safety population was defined as all randomized patients who took at least one dose of the study medication.

Primary Efficacy Analysis

The primary endpoint (change in HbA_{1c} from baseline to week 24) was analyzed using an analysis of covariance (ANCOVA) model with treatment, randomization strata of screening HbA_{1c} (<8.0, ≧8.0%), randomization strata of screening BMI (<30, ≧30 kg/m²) and country as fixed effects and using the baseline value as a covariate.

Differences between lixisenatide and exenatide and two-sided 95% confidence intervals were estimated within the framework of ANCOVA. To assess non-inferiority, the upper bound of the two-sided 95% CI for the difference of the adjusted mean change in HbA_{1c} from baseline to week 24 between lixisenatide and exenatide was compared with the predefined non-inferiority margin of 0.4% HbA_{1c}. Non-inferiority was demonstrated if the upper bound of the two-sided 95% CI of the difference between lixisenatide and exenatide on mITT population was ≦0.4%. If non-inferiority was established, a corresponding check of statistical superiority was to be performed for the primary endpoint.

The primary analysis of the primary efficacy variable was performed based on the mITT population and the measurements obtained during the main 24-week on-treatment period for efficacy variables. The main 24-week on-treatment period was defined as the time from the first dose of the IP up to 3 days (except for FPG by central laboratory, which was up to 1 day) after the last dose of the IP injection on or before V11/week 24 visit (or D169 if V11/week 24 visit was missing), or up to the introduction of rescue therapy, whichever was the earliest. In case of discontinuation of IP before week 24, HbA_{1c} was assessed at the time of discontinuation. The LOCF procedure was used by taking this last available post-baseline on-treatment HbA_{1c} measurement (before the initiation of the new medication in the event of rescue therapy) as the HbA_{1c} value at week 24.

Secondary Efficacy Analysis

No formal statistical test was performed for any secondary efficacy endpoints.

All continuous secondary efficacy variables at week 24 as described in Section 3.2.1 were analyzed using the similar approach and ANCOVA model as described above for the primary analysis of the primary efficacy endpoint. The adjusted estimates of the treatment mean difference between lixisenatide and exenatide and two-sided 95% confidence intervals were provided.

The following categorical secondary efficacy variables at the week 24 were analyzed:
Percentage of patients with HbA_{1c}<7.0% at week 24;
Percentage of patients with HbA_{1c}≦6.5% at week 24;
Percentage of patients requiring rescue therapy during main 24-week treatment period.

Number and percentage of patients with ≧5% weight loss from baseline at week 24 were presented by treatment groups.

All secondary endpoints at the end of treatment were only evaluated by descriptive statistics (mean, standard deviation, median and ranges provided in CSR)

Safety Analysis

The safety analyses were primarily based on the on-treatment period of the whole study. The on-treatment period of the whole study was defined as the time from the first dose of open-label IP injection up to 3 days after the last dose of open-label IP administration during the whole study period regardless of rescue status. The 3-day interval was chosen based on the half-life of the IP (approximately 5 times the half-life).

In addition, the safety analyses for the 24-week treatment period will be summarized in CSR. The summary of safety results (descriptive statistics or frequency tables) is presented by treatment groups.

Health Related Quality-of-Life Analysis

No formal statistical test was performed for the PAGI-QOL total score.

The PAGI-QOL total score at week 24 was analyzed using the similar approach and ANCOVA model as described above for the primary analysis of the primary efficacy endpoint.

Results

Study Patients

Patient Accountability

The study was conducted in 122 centers in 18 countries (Argentina, Austria, Brazil, Colombia, Denmark, Finland, Germany, Greece, Hungary, Italy, Netherlands, Norway, Poland, Puerto Rico, Russian Federation, Spain, Sweden and United States). A total of 1243 patients were screened and 639 were randomized to one of the two treatment groups. One site in Germany (#276905) who randomized 5 patients (out of 8 screened patients) was found to have a significant non-compliance to the protocol. Prior to the database lock, it was decided to exclude these patients from all efficacy and safety analyses and subsequently this had been communicated to US FDA. Safety data from this site will be separately reported in the CSR. The main reason for screening failure was HbA_{1c} value at the screening visit out of the defined protocol ranges (426 [34.5%] out of 1235 screened patients excluding the German site mentioned above).

Six hundred thirty-four randomized patients were included in the analysis (318 in the lixisenatide group and 316 in the exenatide group) and all patients were exposed to the study treatment. Eighteen patients (7 patients in the lixisenatide group and 11 patients in the exenatide group) were excluded from mITT population for efficacy analyses due to lack of post-baseline efficacy data. Table 1 provides the number of patients included in each analysis population.

TABLE 1
Analysis populations-Randomized population

| | Lixisenatide | Exenatide | All |
|---|---|---|---|
| | (N = 318) | (N = 316) | (N = 634) |
| Randomized population | 318 (100%) | 316 (100%) | 634 (100%) |
| Efficacy populations | | | |
| Modified Intent-to-Treat (mITT) | 311 (97.8%) | 305 (96.5%) | 616 (97.2%) |
| Randomized population | 318 (100%) | 316 (100%) | 634 (100%) |
| Safety population | 318 (100%) | 316 (100%) | 634 (100%) |

Note:
The safety population patients are tabulated according to treatment actually received (as treated).
For the efficacy population, patients are tabulated according to their randomized treatment (as randomized).

Study Disposition

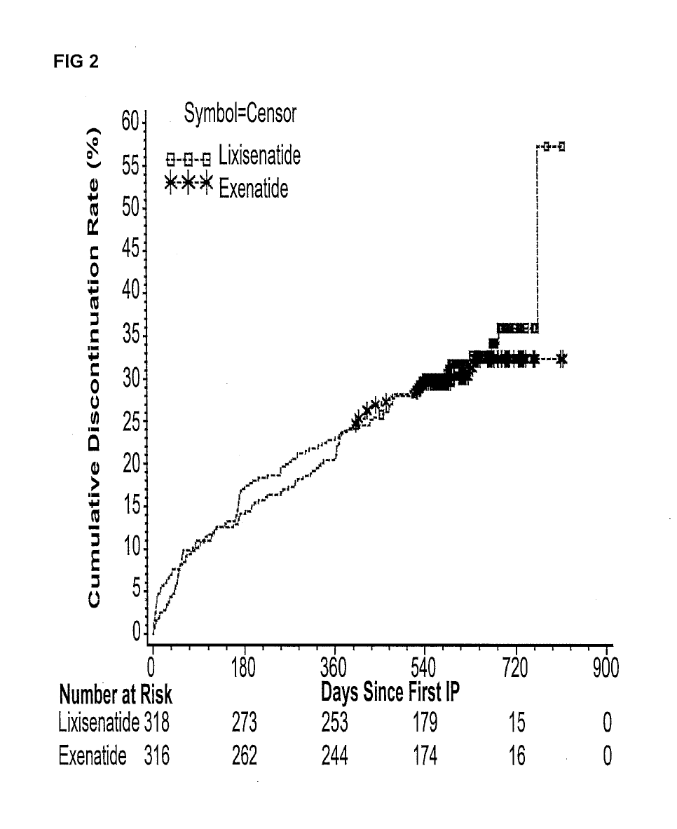

Table 2 provides the summary of patient disposition for each treatment group. During the overall treatment period, 198 (31.2%) patients prematurely discontinued the study treatment. The percentages of patients who discontinued the treatment were similar between treatment groups (32.1% for lixisenatide and 30.4% for exenatide). The main reason for treatment discontinuation was “adverse events” (14.2% in each group) followed by “other reasons” (9.1% for lixisenatide and 9.8% for exenatide), “lack of efficacy” (6.0% for lixisenatide and 1.9% for exenatide) and “poor compliance to protocol” (2.2% for lixisenatide and 4.1% for exenatide). The time-to-onset of treatment discontinuation due to any reason for the overall treatment period is depicted in FIG. 2 with no difference between the 2 treatment groups being observed. Similar results were observed for the 24-week treatment period, where a total of 86 (13.6%) patients prematurely discontinued the study treatment with the main reason also being adverse events (9.1% for lixisenatide and 9.8% for exenatide).

TABLE 2

| | Patient disposition-Randomized population | | | |
|---|---|---|---|---|
| | Lixisenatide | | Exenatide | |
| | (N = 318) | | (N = 316) | |
| Randomized and treated | 318 | (100%) | 316 | (100%) |
| Did not complete 24-week | 41 | (12.9%) | 45 | (14.2%) |
| study treatment | | | | |
| Subject's request for | 34 | (10.7%) | 39 | (12.3%) |
| 24-week treatment | | | | |
| discontinuation | | | | |
| Randomized and treated | 318 | (100%) | 316 | (100%) |
| Reason for 24-week study | 41 | (12.9%) | 45 | (14.2%) |
| treatment discontinuation | | | | |
| Adverse event | 29 | (9.1%) | 31 | (9.8%) |
| Lack of efficacy | 5 | (1.6%) | 1 | (0.3%) |
| Poor compliance to | 0 | | 8 | (2.5%) |
| protocol | | | | |
| Lost to follow-up | 0 | | 0 | |
| Other reasons | 7 | (2.2%) | 5 | (1.6%) |
| Did not complete the | 102 | (32.1%) | 96 | (30.4%) |
| study treatment | | | | |
| Subject's request for | | | | |
| treatment discontinuation | 70 | (22.0%) | 69 | (21.8%) |
| Reason for study | 102 | (32.1%) | 96 | (30.4%) |
| treatment | | | | |
| discontinuation | | | | |
| Adverse event | 45 | (14.2%) | 45 | (14.2%) |
| Lack of efficacy | 19 | (6.0%) | 6 | (1.9%) |
| Poor compliance to | 7 | (2.2%) | 13 | (4.1%) |
| protocol | | | | |
| Lost to follow-up | 2 | (0.6%) | 1 | (0.3%) |
| Other reasons | 29 | (9.1%) | 31 | (9.8%) |
| Status at last study | 318 | (100%) | 316 | (100%) |
| contact | | | | |
| Alive | 311 | (97.8%) | 311 | (98.4%) |
| Dead | 4 | (1.3%) | 4 | (1.3%) |
| Lost to follow-up | 3 | (0.9%) | 1 | (0.3%) |

Note:
Percentages are calculated using the number of randomized patients as denominator.

Demographics and Baseline Characteristics

The demographic and patient baseline characteristics were generally similar between the two treatment groups for the safety population (Table 3). The median age of the study population was 57.5 years. The majority of the patients were Caucasian (92.7%). The percentage of male patients (59.2%) in the exenatide group was higher than the percentage in the lixisenatide group (47.5%).

TABLE 3
Demographics and patient characteristics at screening or baseline-
Safety population

| | Lixisenatide | | Exenatide | | All | |
|---|---|---|---|---|---|---|
| | (N = 318) | | (N = 316) | | (N = 634) | |
| Age (years) | | | | | | |
| Number | 318 | | 316 | | 634 | |
| Mean (SD) | 57.3 | (9.2) | 57.6 | (10.7) | 57.4 | (9.9) |
| Median | 57.0 | | 58.0 | | 57.5 | |
| Min:Max | 29:84 | | 21:83 | | 21:84 | |
| Age group (years) | | | | | | |
| [n (%)] | | | | | | |
| Number | 318 | | 316 | | 634 | |
| <50 | 59 | (18.6%) | 74 | (23.4%) | 133 | (21.0%) |
| ≧50 to <65 | 191 | (60.1%) | 165 | (52.2%) | 356 | (56.2%) |
| ≧65 to <75 | 59 | (18.6%) | 62 | (19.6%) | 121 | (19.1%) |
| ≧75 | 9 | (2.8%) | 15 | (4.7%) | 24 | (3.8%) |
| Gender [n (%)] | | | | | | |
| Number | 318 | | 316 | | 634 | |
| Male | 151 | (47.5%) | 187 | (59.2%) | 338 | (53.3%) |
| Female | 167 | (52.5%) | 129 | (40.8%) | 296 | (46.7%) |
| Age (years) | | | | | | |
| Race [n (%)] | | | | | | |
| Number | 318 | | 316 | | 634 | |
| Caucasian/White | 296 | (93.1%) | 292 | (92.4%) | 588 | (92.7%) |
| Black | 8 | (2.5%) | 10 | (3.2%) | 18 | (2.8%) |
| Asian/Oriental | 3 | (0.9%) | 4 | (1.3%) | 7 | (1.1%) |
| Other | 11 | (3.5%) | 10 | (3.2%) | 21 | (3.3%) |
| Ethnicity [n (%)] | | | | | | |
| Number | 318 | | 316 | | 634 | |
| Hispanic | 87 | (27.4%) | 83 | (26.3%) | 170 | (26.8%) |
| Not Hispanic | 231 | (72.6%) | 233 | (73.7%) | 464 | (73.2%) |
| Screening HbA1c (%) | | | | | | |
| Number | 318 | | 316 | | 634 | |
| Mean (SD) | 8.03 | (0.80) | 8.02 | (0.78) | 8.02 | (0.79) |
| Median | 7.90 | | 7.90 | | 7.90 | |
| Min:Max | 7.0:10.0 | | 7.0:10.0 | | 7.0:10.0 | |
| Randomized strata of | | | | | | |
| screening HbA1c (%) | | | | | | |
| [n (%)] | | | | | | |
| Number | 318 | | 316 | | 634 | |
| <8 | 169 | (53.1%) | 169 | (53.5%) | 338 | (53.3%) |
| ≧8 | 149 | (46.9%) | 147 | (46.5%) | 296 | (46.7%) |
| Age (years) | | | | | | |
| Screening BMI (kg/m²) | | | | | | |
| Number | 318 | | 316 | | 634 | |
| Mean (SD) | 33.68 | (6.28) | 33.51 | (6.53) | 33.60 | (6.40) |
| Median | 32.64 | | 32.50 | | 32.61 | |
| Min:Max | 21.3:54.7 | | 21.4:69.4 | | 21.3:69.4 | |
| Randomized strata of | | | | | | |
| screening BMI | | | | | | |
| Categories (kg/m²) | | | | | | |
| [n (%)] | | | | | | |
| Number | 318 | | 316 | | 634 | |
| <30 | 108 | (34.0%) | 108 | (34.2%) | 216 | (34.1%) |
| ≧30 | 210 | (66.0%) | 208 | (65.8%) | 418 | (65.9%) |
| Baseline BMI (kg/m²) | | | | | | |
| Number | 318 | | 316 | | 634 | |
| Mean (SD) | 33.68 | (6.27) | 33.51 | (6.54) | 33.60 | (6.40) |
| Median | 32.72 | | 32.48 | | 32.58 | |
| Min:Max | 21.5:54.9 | | 21.2:69.3 | | 21.2:69.3 | |
| Baseline BMI | | | | | | |
| Categories (kg/m²) | | | | | | |
| [n (%)] | | | | | | |
| Number | 318 | | 316 | | 634 | |
| <30 | 102 | (32.1%) | 109 | (34.5%) | 211 | (33.3%) |
| ≧30 | 216 | (67.9%) | 207 | (65.5%) | 423 | (66.7%) |

BMI = Body Mass Index.

Disease characteristics including diabetic history were generally comparable between the two treatment groups (Table 4). The mean duration of metformin treatment was slightly longer in the exenatide group (4.21 years) than in the lixisenatide group (3.79 years).

TABLE 4
Disease characteristics at screening or baseline - Safety population

| | Lixisenatide | | Exenatide | | AII | |
|---|---|---|---|---|---|---|
| | (N = 318) | | (N = 316) | | (N = 634) | |
| Duration of diabetes (years) | | | | | | |
| Number | 318 | | 316 | | 634 | |
| Mean (SD) | 6.78 | (5.54) | 6.75 | (4.87) | 6.76 | (5.21) |
| Median | 5.56 | | 5.76 | | 5.68 | |
| Min:Max | 0.9:43.1 | | 1.1:34.8 | | 0.9:43.1 | |
| Age at onset of type 2 diabetes(years) | | | | | | |
| Number | 318 | | 316 | | 634 | |
| Mean (SD) | 50.53 | (9.63) | 50.85 | (10.28) | 50.69 | (9.95) |
| Median | 51.00 | | 52.00 | | 51.00 | |
| MM:Max | 16.0:72.0 | | 18.0:77.0 | | 16.0:77.0 | |
| Duration of metformin treatment | | | | | | |
| (years) | | | | | | |
| Number | 317 | | 316 | | 633 | |
| Mean (SD) | 3.79 | (3.58) | 4.21 | (3.89) | 4.00 | (3.74) |
| Median | 2.49 | | 2.90 | | 2.74 | |
| MM:Max | 0.2:25.1 | | 0.3:27.3 | | 0.2:27.3 | |
| Daily dose of metformin at baseline | | | | | | |
| (mg) | | | | | | |
| Number | 318 | | 316 | | 634 | |
| Mean (SD) | 2020.20 | (459.41) | 2058.39 | (453.23) | 2039.24 | (456.38) |
| Median | 2000.00 | | 2000.00 | | 2000.00 | |
| Min:Max | 1500.0:3000.0 | | 1500.0:3000.0 | | 1500.0:3000.0 | |
| Categorized daily dose of metformin at | | | | | | |
| baseline (mg) [n (%)] | | | | | | |
| Number | 318 | | 316 | | 634 | |
| <1500 | 0 | | 0 | | 0 | |
| ≧1500-<2500 | 231 | (72.6%) | 225 | (71.2%) | 456 | (71.9%) |
| ≧2500-<3000 | 61 | (19.2%) | 62 | (19.6%) | 123 | (19.4%) |
| ≧3000 | 26 | (8.2%) | 29 | (9.2%) | 55 | (8.7%) |
| History of gestational diabetes | | | | | | |
| Number (Female) | 167 | | 129 | | 296 | |
| Yes (Female) | 11 | (6.6%) | 14 | (10.9%) | 25 | (8.4%) |
| No (Female) | 156 | (93.4%) | 115 | (89.1%) | 271 | (91.6%) |
| Prior use of GLP-1 receptor agonist | | | | | | |
| [n (%)] | | | | | | |
| Number | 318 | | 316 | | 634 | |
| Yes | 4 | (1.3%) | 7 | (2.2%) | 11 | (1.7%) |
| No | 314 | (98.7%) | 309 | (97.8%) | 623 | (98.3%) |
| Diabetic retinopathy [n (%)] | | | | | | |
| Number | 317 | | 314 | | 631 | |
| Yes | 24 | (7.6%) | 12 | (3.8%) | 36 | (5.7%) |
| No | 283 | (89.3%) | 281 | (89.5%) | 564 | (89.4%) |
| Unknown | 10 | (3.2%) | 21 | (6.7%) | 31 | (4.9%) |
| Diabetic sensory or motor neuropathy | | | | | | |
| [n (%)] | | | | | | |
| Number | 317 | | 314 | | 631 | |
| Yes | 33 | (10.4%) | 39 | (12.4%) | 72 | (11.4%) |
| No | 274 | (86.4%) | 264 | (84.1%) | 538 | (85.3%) |
| Unknown | 10 | (3.2%) | 11 | (3.5%) | 21 | (3.3%) |
| Diabetic autonomic neuropathy [n (%)] | | | | | | |
| Number | 317 | | 314 | | 631 | |
| Yes | 2 | (0.6%) | 0 | | 2 | (0.3%) |
| No | 303 | (95.6%) | 299 | (95.2%) | 602 | (95.4%) |
| Unknown | 12 | (3.8%) | 15 | (4.8%) | 27 | (4.3%) |
| Diabetic nephropathy [n (%)] | | | | | | |
| Number | 317 | | 314 | | 631 | |
| Yes | 7 | (2.2%) | 12 | (3.8%) | 19 | (3.0%) |
| Microalbuminuria | 6 | (1.9%) | 8 | (2.5%) | 14 | (2.2%) |
| Overt proteinuria | 0 | | 1 | (0.3%) | 1 | (0.2%) |
| Impaired renal function | 1 | (0.3%) | 3 | (1.0%) | 4 | (0.6%) |
| Dialysis or transplantation | 0 | | 0 | | 0 | |
| No | 299 | (94.3%) | 284 | (90.4%) | 583 | (92.4%) |
| Unknown | 11 | (3.5%) | 18 | (5.7%) | 29 | (4.6%) |
| Creatinine clearance (ml/min) at | | | | | | |
| screening | | | | | | |
| Number | 318 | | 316 | | 634 | |
| Mean (SD) | 125.55 | (38.54) | 129.45 | (47.32) | 127.49 | (43.15) |
| Median | 121.68 | | 119.74 | | 120.55 | |
| Min:Max | 46.0:262.4 | | 32.4:301.4 | | 32.4:301.4 | |
| Creatinine clearance categories at | | | | | | |
| screening [n (%)] | | | | | | |
| Number | 318 | | 316 | | 634 | |
| <30 ml/min (severe renal | | | | | | |
| impairment) | 0 | | 0 | | 0 | |
| ≧30-<50 ml/min (moderate renal | | | | | | |
| impairment) | 3 | (0.9%) | 4 | (1.3%) | 7 | (1.1%) |
| ≧50-≦80 ml/min (mild renal | | | | | | |
| impairment) | 30 | (9.4%) | 35 | (11.1%) | 65 | (10.3%) |
| >80 ml/min (no renal impairment) | 285 | (89.6%) | 277 | (87.7%) | 562 | (88.6%) |

GLP-1 = Glucagon like peptide-1.
Creatinine clearance value is derived using the equation of Cockcroft and Gault.

HbA_{1c} and FPG at baseline were comparable between two treatment groups for the safety population (Table 5). A higher mean body weight at baseline was observed in the exenatide group (96.09 kg) compared with the lixisenatide group (94.01 kg).

TABLE 5
Baseline efficacy variables Safety population

| | Lixisenatide | | Exenatide | | AII | |
|---|---|---|---|---|---|---|
| | (N = 318) | | (N = 316) | | (N = 634) | |
| HbA1c (%) | | | | | | |
| Number | 318 | | 316 | | 634 | |
| Mean (SD) | 7.95 | (0.81) | 7.97 | (0.78) | 7.96 | (0.80) |
| Median | 7.80 | | 7.90 | | 7.80 | |
| MM:Max | 6.1:10.2 | | 6.1:9.9 | | 6.1:10.2 | |
| Weight (kg) | | | | | | |
| Number | 318 | | 316 | | 634 | |
| Mean (SD) | 94.01 | (19.63) | 96.09 | (22.52) | 95.04 | (21.13) |
| Median | 92.45 | | 92.30 | | 92.35 | |
| MM:Max | 51.3:176.0 | | 51.2:192.8 | | 51.2:192.8 | |
| FPG (mmol/L) | | | | | | |
| Number | 318 | | 316 | | 634 | |
| Mean (SD) | 9.68 | (2.03) | 9.66 | (2.26) | 9.67 | (2.15) |

| | | | |
|---|---|---|---|
| Median | 9.30 | 9.30 | 9.30 |
| Min:Max | 5.7:15.4 | 4.1:18.9 | 4.1:18.9 |

FPG = Fasting Plasma Glucose.

The patient assessment of upper gastrointestinal disorders-Quality of life (PAGI-QOL) total score at baseline was similar between the two treatment groups (Table 6).

TABLE 6
Baseline patient assessment of upper gastrointestinal disorders -
Quality of life (PAGI-QOL) - Safety population

| | Lixisenatide | Exenatide | AII |
|---|---|---|---|
| | (N = 318) | (N = 316) | (N = 634) |
| PAGI-QOL total score | | | |
| Number | 314 | 313 | 627 |
| Mean (SD) | 0.59 (0.72) | 0.56 (0.72) | 0.58 (0.72) |
| Median | 0.27 | 0.27 | 0.27 |
| Min:Max | 0.0:3.1 | 0.0:3.5 | 0.0:3.5 |

Dosage and Duration

The average treatment exposure was similar between the two treatment groups (494.8 days (70.6 weeks) for the lixisenatide group and 483.0 days (69 weeks) for the exenatide group) [Table 7]. Out of 634 patients, 536 (85.2% in the lixisenatide group and 83.9% in the exenatide group) had at least 169 days (24 weeks) of treatment and 345 (55.0% in the lixisenatide group and 53.8% in the exenatide group) had at least 547 days (18 months) of treatment. Note that the treatment duration of 5 patients (4 patients in the lixisenatide group and 1 patient in the exenatide group) was not summarized due to their missing end of treatment dates.

For the lixisenatide group, 295 (92.8%) patients and 293 (92.1%) patients were at the target total daily dose of 20 μg at the end of the 24-week treatment period and at the end of treatment, respectively (Tables 8 and 9). For the exenatide group, 263 (83.2%) patients and 217 (68.7%) patients were at the target total daily dose 20 μg at the end of 24-week treatment period and at the end of treatment, respectively (Tables 8 and 9).

TABLE 7
Exposure - Safety population

| | Lixisenatide | | Exenatide | |
|---|---|---|---|---|
| | (N = 318) | | (N = 316) | |
| Cumulative duration of treatment | 425.4 | | 416.6 | |
| exposure (patient years) | | | | |
| Duration of study treatment (days) | | | | |
| Number | 314 | | 315 | |
| Mean (SD) | 494.8 | (206.1) | 483.0 | (216.9) |
| Median | 562.0 | | 560.0 | |
| Min:Max | 1:814 | | 1:815 | |
| Duration of study treatment by | | | | |
| category [n (%)] | | | | |
| 1-14 days | 8 | (2.5%) | 16 | (5.1%) |
| 15-28 days | 3 | (0.9%) | 4 | (1.3%) |
| 29-56 days | 15 | (4.7%) | 6 | (1.9%) |
| 57-84 days | 7 | (2.2%) | 6 | (1.9%) |
| 85-168 days | 10 | (3.1%) | 18 | (5.7%) |
| 169-364 days | 24 | (7.5%) | 23 | (7.3%) |
| 365-546 days | 72 | (22.6%) | 72 | (22.8%) |
| 547-728 days | 163 | (51.3%) | 157 | (49.7%) |
| >728 days | 12 | (3.8%) | 13 | (4.1%) |
| Cumulative duration of study treatment | | | | |
| by category [n (%)] | | | | |
| ≧1 day | 314 | (98.7%) | 315 | (99.7%) |
| ≧15 days | 306 | (96.2%) | 299 | (94.6%) |
| ≧29 days | 303 | (95.3%) | 295 | (93.4%) |
| ≧57 days | 288 | (90.6%) | 289 | (91.5%) |
| ≧85 days | 281 | (88.4%) | 283 | (89.6%) |
| ≧169 days | 271 | (85.2%) | 265 | (83.9%) |
| ≧365 days | 247 | (77.7%) | 242 | (76.6%) |
| ≧547 days | 175 | (55.0%) | 170 | (53.8%) |
| ≧729 days | 12 | (3.8%) | 13 | (4.1%) |

Note:
Duration of exposure = (date of the last IP injection - date of the first IP injection) + 1.

TABLE 8
Number (%) of patients by final total daily dose at the end
of the 24-week treatment - Safety population

| Dose at the end of the 24- | Lixisenatide | | Exenatide | |
|---|---|---|---|---|
| week | (N = 318) | | (N = 316) | |
| <10 μg | 0 | | 10 | (3.2%) |
| 10 μg | 11 | (3.5%) | 43 | (13.6%) |
| 15 μg | 12 | (3.8%) | 0 | |
| 20 μg | 295 | (92.8%) | 263 | (83.2%) |

Note:
Percents are calculated using the number of safety patients as the denominator.

TABLE 9
Number (%) of patients by final total daily dose at the end
of the treatment - Safety population

| | Lixisenatide | | Exenatide | |
|---|---|---|---|---|
| Final Dose | (N = 318) | | (N = 316) | |
| <10 μg | 0 | | 12 | (3.8%) |
| 10 μg | 15 | (4.7%) | 87 | (27.5%) |
| 15 μg | 10 | (3.1%) | 0 | |
| 20 μg | 293 | (92.1%) | 217 | (68.7%) |

Note:
Percents are calculated using the number of safety patients as the denominator.

Efficacy

Primary Efficacy Endpoint

### Main Analysis

Table 10 summarizes the results of the primary efficacy parameter, change from baseline to Week 24 (LOCF) in HbA_{1c} using an ANCOVA analysis.

The LS mean changes from baseline to Week 24 in HbA_{1c} was −0.79% for the lixisenatide group and −0.96% for the exenatide group (LS mean difference versus exenatide=0.17%). Based on the pre-specified primary analysis, non-inferiority of lixisenatide versus exenatide was demonstrated as the upper bound of the two-sided 95% CI of the LS mean difference was less than the predefined non-inferiority margin 0.4%. Superiority of lixisenatide over exenatide was not demonstrated.

TABLE 10
Mean change in HbA1c (%) from baseline to week 24 - mITT population

| | Lixisenatide | | Exenatide | |
|---|---|---|---|---|
| HbAlc (%) | (N = 311) | | (N = 305) | |
| Baseline | | | | |
| Number | 295 | | 297 | |
| Mean (SD) | 7.97 | (0.82) | 7.96 | (0.77) |
| Median | 7.80 | | 7.90 | |
| Min:Max | 6.1:10.2 | | 6.1:9.9 | |
| Week 24 (LOCF) | | | | |
| Number | 295 | | 297 | |
| Mean (SD) | 7.17 | (0.96) | 7.01 | (0.88) |
| Median | 7.00 | | 7.00 | |
| Min:Max | 5.3:11.1 | | 4.9:10.7 | |
| Change from baseline to week 24 (LOCF) | | | | |
| Number | 295 | | 297 | |
| Mean (SD) | −0.80 | (0.88) | −0.95 | (0.87) |
| Median | −0.80 | | −0.90 | |
| MM:Max | −3.1:3.8 | | −3.3:3.4 | |
| LS Mean (SE) a | −0.79 | (0.053) | −0.96 | (0.054) |

| | | |
|---|---|---|
| LS Mean difference (SE) vs. Exenatide^{a} | 0.17 | (0.067) |

| | |
|---|---|
| 95% CI | (0.033 to 0.297) |

LOCF = Last observation carry forward.
^{a}Analysis of covariance (ANCOVA) model with treatment groups (Exenatide and Lixisenatide), randomization strata of screening HbAlc (<8.0, ≧8.0%), randomization strata of screening BMI (<30, ≧30 kg/m²), and country as fixed effects and baseline HbAlc value as a covariate.
Note:
The analysis included measurements obtained before the introduction of rescue medication and up to 3 days after the last dose of the investigational product injection on or before Visit 11 (Week24), or Day 169 if Visit 11 (Week 24) is not available.
Patients with both baseline and Week 24 (LOCF) measurements are included.

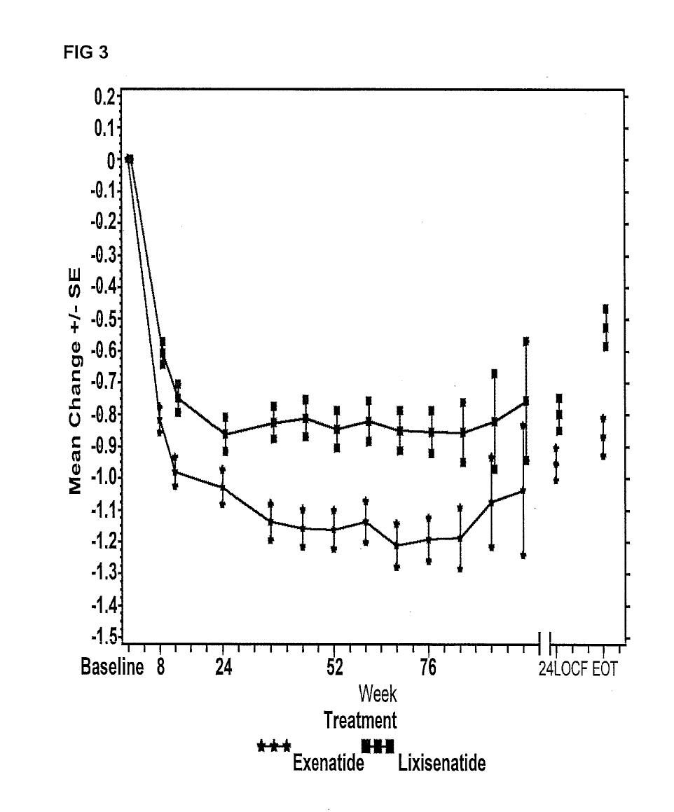

FIG. 3 illustrates the Mean (±SE) change from baseline in HbA1c over time during the whole treatment period (up to 2 years shown). The HbA1c reduction was relatively maintained over time beyond 24 weeks.

### Secondary Analysis

Table 11 summarizes the proportion of patients with treatment response HbA_{1c}≦6.5% or <7% at Week 24, respectively. At Week 24, 28.5% of lixisenatide-treated patients and 35.4% of exenatide-treated patients had achieved HbA_{1c} values ≦6.5%; 48.5% of patients in the lixisenatide group and 49.8% of patients in the exenatide group had achieved HbA_{1c} values <7%.

TABLE 11
Number (%) of patients with HbA_{1c} value ≦6.5% or <7% respectively
at week 24 - mITT population

| | Lixisenatide | | Exenatide | |
|---|---|---|---|---|
| HbA1c (%) | (N = 311) | | (N = 305) | |
| Number | 295 | | 297 | |
| ≦6.5% | 84 | (28.5%) | 105 | (35.4%) |
| Number | 295 | | 297 | |
| <7.0% | 143 | (48.5%) | 148 | (49.8%) |

Note:
The analysis included measurements obtained before the introduction of rescue medication and up to 3 days after the last dose of the investigational product injection on or before Visit 11 (Week 24), or Day 169 if Visit 11 (Week 24) is not available.

Other Efficacy Endpoints

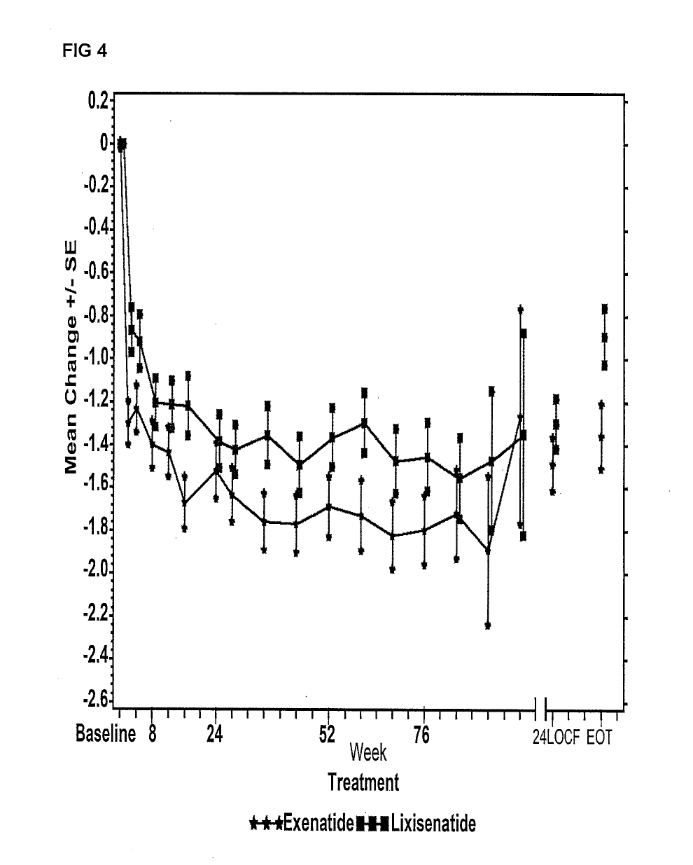

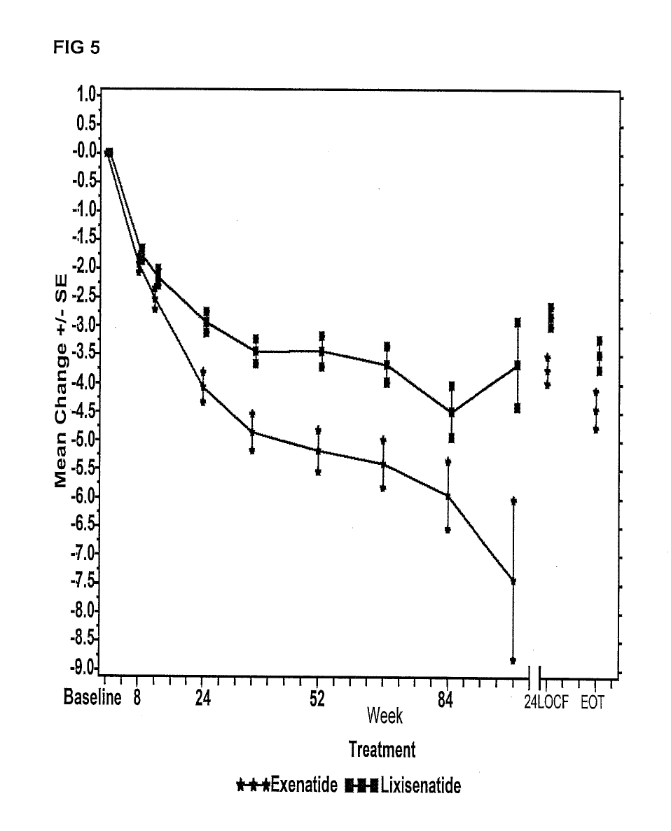

Table 12 and Table 13 summarize the ANCOVA analyses of FPG and body weight, respectively. FIG. 4 and FIG. 5 illustrate the Mean (±SE) change from baseline in FPG and body weight over time during the whole treatment period (up to 2 years shown).

For FPG, the LS mean changes from baseline to Week 24 was −1.22 mmol/L for the lixisenatide group and −1.45 mmol/L for the exenatide group (LS mean difference versus exenatide=0.23 mmol/L).

The LS mean body weight loss from baseline at Week 24 was 2.96 kg for the lixisenatide-treated patients and 3.98 kg for the exenatide-treated patients (LS mean difference versus exenatide=1.02 kg). Body weight continued to decrease after the 24 week main treatment period in both treatments (FIG. 5). About 25.1% lixisenatide-treated patients and 31.4% exenatide-treated patients had >=5% weight loss from baseline to week 24 (Table 14).

The percentages of patients requiring rescue therapy at Week 24 were small in the two groups (Table 15).

TABLE 12
Mean change in fasting plasma glucose (mmol/L) from
baseline to week 24 - mITT population

| | Lixisenatide | | Exenatide | |
|---|---|---|---|---|
| Fasting plasma glucose (mmol/L) | (N = 311) | | (N = 305) | |
| Baseline | | | | |
| Number | 310 | | 301 | |
| Mean (SD) | 9.72 | (2.03) | 9.68 | (2.25) |
| Median | 9.40 | | 9.40 | |
| Min:Max | 5.7:15.4 | | 4.1:18.9 | |
| Week 24 (LOCF) | | | | |
| Number | 310 | | 301 | |
| Mean (SD) | 8.42 | (2.03) | 8.20 | (2.13) |
| Median | 8.10 | | 8.00 | |
| Min:Max | 4.3:19.2 | | 4.9:19.2 | |
| Change from baseline to week 24 (LOCF) | | | | |
| Number | 310 | | 301 | |
| Mean (SD) | −1.30 | (2.06) | −1.49 | (2.18) |
| Median | −1.25 | | −1.40 | |
| Min:Max | −8.5:6.8 | | −9.3:6.3 | |
| LS Mean (SE)^{a} | −1.22 | (0.116) | −1.45 | (0.119) |

| | | |
|---|---|---|
| LS Mean difference (SE) vs. Exenatide^{a} | 0.23 | (0.146) |

| | |
|---|---|
| 95% CI | (−0.052 to 0.522) |

LOCF = Last observation carry forward.
^{a}Analysis of covariance (ANCOVA) model with treatment groups (Exenatide and Lixisenatide), randomization strata of screening HbA1c (<8.0, ≧8.0%), BMI (<30, ≧30 kg/m²) at screening, and country as fixed effects and baseline FPG as a covariate.
Note:
The analysis included measurements obtained before the introduction of rescue medication and up to 1 day after the last dose of the investigational product injection on or before Visit 11 (Week 24), or Day 169 if Visit 11 (Week 24) is not available. Patients with both baseline and Week 24 (LOCF) measurements are included.

TABLE 13
Mean change in body weight (kg) from baseline to week 24 - mITT population

| | | | LixisenatideExenatide | |
|---|---|---|---|---|
| Body weight (kg) | (N = 311) | | (N = 305) | |
| Baseline | | | | |
| Number | 295 | | 296 | |
| Mean (SD) | 94.51 | (19.37) | 96.69 | (22.80) |
| Median | 92.50 | | 93.00 | |
| Min:Max | 51.3:176.0 | | 51.2:192.8 | |
| Week 24 (LOCF) | | | | |
| Number | 295 | | 296 | |
| Mean (SD) | 91.68 | (18.92) | 92.93 | (22.33) |
| Median | 89.30 | | 91.00 | |
| Min:Max | 48.0:176.0 | | 50.0:185.3 | |
| Change from baseline to week 24 (LOCF) | | | | |
| Number | 295 | | 296 | |
| Mean (SD) | −2.83 | (2.98) | −3.76 | (4.08) |
| Median | −2.60 | | −3.35 | |
| Min:Max | −13.0:9.8 | | −24.2:5.0 | |
| LS Mean (SE)^{a} | −2.96 | (0.231) | −3.98 | (0.232) |

| | | |
|---|---|---|
| LS Mean difference (SE) vs. Exenatide^{a} | 1.02 | (0.286) |

| | |
|---|---|
| 95% CI | (0.456 to 1.581) |

LOCF = Last observation carry forward.
^{a}Analysis of covariance (ANCOVA) model with treatment groups (Exenatide and Lixisenatide), randomization strata of screening HbA1c (<8.0, ≧8.0%), randomization strata of screening BMI (<30, ≧30 kg/m²), and country as fixed effects and baseline body weight as a covariate.
Note:
The analysis included measurements obtained before the introduction of rescue medication and up to 3 days after the last dose of the investigational product injection on or before Visit 11 (Week 24), or Day 169 if Visit 11 (Week 24) is not available. Patients with both baseline and Week 24 (LOCF) measurements are included.

Table 14
Number (%) of patients with >= 5 % weight loss from baseline to
week 24 - mITT population

| | Lixisenatide | | Exenatide | |
|---|---|---|---|---|
| Weight loss | (N = 311) | | (N = 305) | |
| Number | 295 | | 296 | |
| ≧5% | 74 | (25.1%) | 93 | (31.4%) |
| <5% | 221 | (74.9%) | 203 | (68.6%) |

Note:
The analysis included measurements obtained before the introduction of rescue medication and up to 3 days after the last dose of the investigational product injection on or before Visit 11 (Week 24), or Day 169 if Visit 11 (Week 24) is not available.

TABLE 15
Number (%) of patients requiring rescue therapy during the
24-week treatment period -mITT population

| | Lixisenatide | | Exenatide | |
|---|---|---|---|---|
| Requiring rescue therapy | (N = 311) | | (N = 305) | |
| Number | 311 | | 305 | |
| Yes | 7 | (2.3%) | 11 | (3.6%) |
| No | 304 | (97.7%) | 294 | (96.4%) |

Safety

An overview of the adverse events observed during the on-treatment period of the whole study is provided in Table 16. The proportion of patients who experienced TEAEs was generally comparable between the lixisenatide-treated and exenatide-treated groups. Six patients (3 patients in each treatment group) had SAEs during the on-treatment period leading to death. Forty-eight serious TEAEs occurred during the on-treatment period of the whole study with a similar incidence rate in each treatment group (8.2% for lixisenatide and 7.0% for exenatide). The percentage of patients with TEAEs leading to treatment discontinuation was the same in both groups (14.2%). Tables 17, 18, and 19 summarize TEAEs leading to death, serious TEAEs, and TEAEs leading to treatment discontinuation by primary SOC, HLGT, HLT and PT, respectively. The most common TEAE leading to treatment discontinuation was nausea in both treatment groups (15 [4.7%] patients in lixisenatide and 19 [6.0%] patients in exenatide).

Table 29 in the appendix presents the incidences of TEAEs during the on-treatment period of the whole study occurring in at least 1% of patients in any treatment group. Nausea was the most frequently reported TEAE in the lixisenatide group (91 patients [28.6%]). A higher percentage of exenatide-treated patients (119 [37.7%] patients) reported nausea. The second most frequently reported TEAE in the lixisenatide-treated patients was diarrhea (48 patients [15.1%]) followed by headache (46 patients [14.5%]). The corresponding number of patients (%) in the exenatide group was 54 (17.1%) for diarrhea and 31 (9.8%) for headache.

TABLE 16
Overview of adverse event profile: treatment emergent adverse events
during the on-treatment period for the whole study - Safety population

| | Lixisenatide | | Exenatide | |
|---|---|---|---|---|
| | (N = 318) | | (N = 316) | |
| Patients with any TEAE | 257 | (80.8%) | 264 | (83.5%) |
| Patients with any serious TEAE | 26 | (8.2%) | 22 | (7.0%) |
| Patients with any TEAE leading to death | 3 | (0.9%) | 3 | (0.9%) |
| Patients with any TEAE leading to | 45 | (14.2%) | 45 | (14.2%) |
| permanent treatment discontinuation | | | | |

TEAE: Treatment Emergent Adverse Event
n (%) = number and percentage of patients with at least one adverse event

TABLE 17
Number (%) of patients experiencing TEAE(s) leading to death during the overall
treatment period by primary SOC, HLGT, HLT, and PT - Safety population

| PRIMARY SYSTEM ORGAN CLASS | | | | |
|---|---|---|---|---|
| HLGT: High Level Group Term | | | | |
| HLT: High Level Term | Lixisenatide | | Exenatide | |
| Preferred Term | (N = 318) | | (N = 316) | |
| Any class | 3 | (0.9%) | 3 | (0.9%) |
| INFECTIONS AND INFESTATIONS | 1 | (0.3%) | 0 | |
| HLGT: Infections - pathogen unspecified | 1 | (0.3%) | 0 | |
| HLT: Sepsis, bacteraemia, viraemia and fungaemia NEC | 1 | (0.3%) | 0 | |
| Sepsis | 1 | (0.3%) | 0 | |
| NEOPLASMS BENIGN, MALIGNANT AND UNSPECIFIED | 1 | (0.3%) | 1 | (0.3%) |
| (INCL CYSTS AND POLYPS) | | | | |
| HLGT: Gastrointestinal neoplasms malignant and unspecified | 0 | | 1 | (0.3%) |
| HLT: Pancreatic neoplasms malignant (excl islet cell and | 0 | | 1 | (0.3%) |
| carcinoid) | | | | |
| Pancreatic carcinoma | 0 | | 1 | (0.3%) |
| HLGT: Miscellaneous and site unspecified neoplasms malignant | 1 | (0.3%) | 0 | |
| and unspecified | | | | |
| HLT: Neoplasms malignant site unspecified NEC | 1 | (0.3%) | 0 | |
| Metastatic neoplasm | 1 | (0.3%) | 0 | |
| CARDIAC DISORDERS | 1 | (0.3%) | 2 | (0.6%) |
| HLGT: Coronary artery disorders | 1 | (0.3%) | 2 | (0.6%) |
| HLT: Ischaemic coronary artery disorders | 1 | (0.3%) | 2 | (0.6%) |

| | | | |
|---|---|---|---|
| Acute myocardial infarction | 0 | 1 | (0.3%) |
| Myocardial infarction | 0 | 1 | (0.3%) |

| | | | |
|---|---|---|---|
| Myocardial ischaemia | 1 | (0.3%) | 0 |

TEAS: Treatment Emergent Adverse Event,
SOC: System Organ Class, HLGT: High Level Group Term,
HLT: High Level term, PT: Preferred Term.
MedDRA version: 13.1
Note:
Table sorted by SOC internationally agreed order and HLGT, HLT, PT alphabetic order.

TABLE 18
Number (%) of patients experiencing serious TEAE(s) during the overall treatment
period presented by primary SOC, HLGT, HLT, and PT - Safety population

| PRIMARY SYSTEM ORGAN CLASS | | |
|---|---|---|
| HLGT: High Level Group Term | | |
| HLT: High Level Term | Lixisenatide | Exenatide |
| Preferred Term | (N = 318) | (N = 316) |
| Any class | 26 (8.2%) | 22 (7.0%) |
| INFECTIONS AND INFESTATIONS | 7 (2.2%) | 4 (1.3%) |
| HLGT: Bacterial infectious disorders | 0 | 2 (0.6%) |
| HLT: Bacterial infections NEC | 0 | 2 (0.6%) |
| Cellulitis | 0 | 1 (0.3%) |
| Pneumonia bacterial | 0 | 1 (0.3%) |
| HLGT: Infections - pathogen unspecified | 7 (2.2%) | 2 (0.6%) |
| HLT: Abdominal and gastrointestinal infections | 1 (0.3%) | 1 (0.3%) |
| Appendicitis | 1 (0.3%) | 1 (0.3%) |
| HLT: Lower respiratory tract and lung infections | 2 (0.6%) | 0 |
| Bronchitis | 1 (0.3%) | 0 |
| Pneumonia | 1 (0.3%) | 0 |
| HLT: Sepsis, bacteraemia, viraemia and fungaemia | 2 (0.6%) | 1 (0.3%) |
| NEC | | |
| Sepsis | 2 (0.6%) | 0 |
| Septic shock | 0 | 1 (0.3%) |
| Urosepsis | 0 | 1 (0.3%) |
| HLT: Upper respiratory tract infections | 1 (0.3%) | 0 |
| Upper respiratory tract infection | 1 (0.3%) | 0 |
| HLT: Urinary tract infections | 1 (0.3%) | 0 |
| Pyelonephritis acute | 1 (0.3%) | 0 |
| Any class | 26 (8.2%) | 22 (7.0%) |
| NEOPLASMS BENIGN, MALIGNANT AND UNSPECIFIED | 3 (0.9%) | 3 (0.9%) |
| (INCL CYSTS AND POLYPS) | | |
| HLGT: Endocrine neoplasms malignant and unspecified | 0 | 1 (0.3%) |
| HLT: Endocrine neoplasms malignant and unspecified | 0 | 1 (0.3%) |
| NEC | | |
| Thyroid neoplasm | 0 | 1 (0.3%) |
| HLGT: Gastrointestinal neoplasms malignant and | 1 (0.3%) | 1 (0.3%) |
| unspecified | | |
| HLT: Gastrointestinal neoplasms malignancy | 1 (0.3%) | 0 |
| unspecified NEC | | |
| Gastrointestinal stromal tumour | 1 (0.3%) | 0 |
| HLT: Pancreatic neoplasms malignant (excl islet cell | 0 | 1 (0.3%) |
| and carcinoid) | | |
| Pancreatic carcinoma | 0 | 1 (0.3%) |
| HLGT: Miscellaneous and site unspecified neoplasms | 1 (0.3%) | 0 |
| malignant and unspecified | | |
| HLT: Neoplasms malignant site unspecified NEC | 1 (0.3%) | 0 |
| Metastatic neoplasm | 1 (0.3%) | 0 |
| HLGT: Reproductive neoplasms male malignant and | 0 | 1 (0.3%) |
| unspecified | | |
| HLT: Prostatic neoplasms malignant | 0 | 1 (0.3%) |
| Prostate cancer | 0 | 1 (0.3%) |
| HLGT: Respiratory and mediastinal neoplasms benign (excl | 1 (0.3%) | 0 |
| mesotheliomas) | | |
| HLT: Respiratory tract and pleural neoplasms benign | 1 (0.3%) | 0 |
| NEC | | |
| Benign lung neoplasm | 1 (0.3%) | 0 |
| Any class | 26 (8.2%) | 22 (7.0%) |
| PSYCHIATRIC DISORDERS | 1 (0.3%) | 1 (0.3%) |
| HLGT: Anxiety disorders and symptoms | 1 (0.3%) | 0 |
| HLT: Anxiety symptoms | 1 (0.3%) | 0 |
| Anxiety | 1 (0.3%) | 0 |
| HLGT: Suicidal and self-injurious behaviours NEC | 0 | 1 (0.3%) |
| HLT: Suicidal and self-injurious behaviour | 0 | 1 (0.3%) |
| Suicide attempt | 0 | 1 (0.3%) |
| NERVOUS SYSTEM DISORDERS | 3 (0.9%) | 4 (1.3%) |
| HLGT: Central nervous system vascular disorders | 1 (0.3%) | 0 |
| HLT: Central nervous system vascular disorders NEC | 1 (0.3%) | 0 |
| Carotid artery stenosis | 1 (0.3%) | 0 |
| HLGT: Cranial nerve disorders (excl neoplasms) | 0 | 1 (0.3%) |
| HLT: Facial cranial nerve disorders | 0 | 1 (0.3%) |
| Facial paresis | 0 | 1 (0.3%) |
| HLGT: Mental impairment disorders | 0 | 1 (0.3%) |
| HLT: Mental impairment (excl dementia and memory | 0 | 1 (0.3%) |
| loss) | | |
| Cognitive disorder | 0 | 1 (0.3%) |
| HLGT: Neurological disorders NEC | 1 (0.3%) | 2 (0.6%) |
| HLT: Disturbances in consciousness NEC | 1 (0.3%) | 2 (0.6%) |
| Loss of consciousness | 0 | 1 (0.3%) |
| Syncope | 1 (0.3%) | 1 (0.3%) |
| HLGT: Spinal cord and nerve root disorders | 1 (0.3%) | 0 |
| HLT: Lumbar spinal cord and nerve root disorders | 1 (0.3%) | 0 |
| Sciatica | 1 (0.3%) | 0 |
| Any class | 26 (8.2%) | 22 (7.0%) |
| EYE DISORDERS | 1 (0.3%) | 0 |
| HLGT: Retina, choroid and vitreous haemorrhages and | 1 (0.3%) | 0 |
| vascular disorders | | |
| HLT: Retinopathies NEC | 1 (0.3%) | 0 |
| Retinopathy | 1 (0.3%) | 0 |
| CARDIAC DISORDERS | 3 (0.9%) | 3 (0.9%) |
| HLGT: Cardiac arrhythmias | 2 (0.6%) | 1 (0.3%) |
| HLT: Rate and rhythm disorders NEC | 1 (0.3%) | 0 |
| Arrhythmia | 1 (0.3%) | 0 |
| HLT: Supraventricular arrhythmias | 1 (0.3%) | 1 (0.3%) |
| Atrial fibrillation | 1 (0.3%) | 1 (0.3%) |
| HLGT: Coronary artery disorders | 1 (0.3%) | 2 (0.6%) |
| HLT: Ischaemic coronary artery disorders | 1 (0.3%) | 2 (0.6%) |
| Acute myocardial infarction | 0 | 1 (0.3%) |
| Myocardial infarction | 0 | 1 (0.3%) |
| Myocardial ischaemia | 1 (0.3%) | 0 |
| VASCULAR DISORDERS | 2 (0.6%) | 2 (0.6%) |
| HLGT: Arteriosclerosis, stenosis, vascular insufficiency and | 1 (0.3%) | 0 |
| necrosis | | |
| HLT: Peripheral vasoconstriction, necrosis and vascular | 1 (0.3%) | 0 |
| insufficiency | | |
| Subclavian artery stenosis | 1 (0.3%) | 0 |
| Any class | 26 (8.2%) | 22 (7.0%) |
| HLGT: Decreased and nonspecific blood pressure disorders | 0 | 2 (0.6%) |
| and shock | | |
| HLT: Vascular hypotensive disorders | 0 | 2 (0.6%) |
| Hypotension | 0 | 2 (0.6%) |
| HLGT: Vascular haemorrhagic disorders | 1 (0.3%) | 0 |
| HLT: Haemorrhages NEC | 1 (0.3%) | 0 |
| Haematoma | 1 (0.3%) | 0 |
| GASTROINTESTINAL DISORDERS | 3 (0.9%) | 0 |
| HLGT: Abdominal hernias and other abdominal wall | 2 (0.6%) | 0 |
| conditions | | |
| HLT: Abdominal hernias, site unspecified | 1 (0.3%) | 0 |
| Abdominal hernia | 1 (0.3%) | 0 |
| HLT: Inguinal hernias | 1 (0.3%) | 0 |
| Inguinal hernia | 1 (0.3%) | 0 |
| HLGT: Gastrointestinal vascular conditions | 1 (0.3%) | 0 |
| HLT: Haemorrhoids and gastrointestinal varices (excl | 1 (0.3%) | 0 |
| oesophageal) | | |
| Haemorrhoids | 1 (0.3%) | 0 |
| HEPATOBILIARY DISORDERS | 1 (0.3%) | 2 (0.6%) |
| HLGT: Gallbladder disorders | 1 (0.3%) | 2 (0.6%) |
| HLT: Cholecystitis and cholelithiasis | 1 (0.3%) | 2 (0.6%) |
| Cholecystitis | 0 | 1 (0.3%) |
| Cholecystitis acute | 1 (0.3%) | 0 |
| Cholecystitis chronic | 0 | 1 (0.3%) |
| Any class | 26 (8.2%) | 22 (7.0%) |
| SKIN AND SUBCUTANEOUS TISSUE DISORDERS | 1 (0.3%) | 0 |
| HLGT: Angioedema and urticaria | 1 (0.3%) | 0 |
| HLT: Urticarias | 1 (0.3%) | 0 |
| Urticaria | 1 (0.3%) | 0 |
| MUSCULOSKELETAL AND CONNECTIVE TISSUE | 0 | 1 (0.3%) |
| DISORDERS | | |
| HLGT: Musculoskeletal and connective tissue disorders | 0 | 1 (0.3%) |
| NEC | | |
| HLT: Musculoskeletal and connective tissue pain and | 0 | 1 (0.3%) |
| discomfort | | |
| Musculoskeletal chest pain | 0 | 1 (0.3%) |
| REPRODUCTIVE SYSTEM AND BREAST DISORDERS | 0 | 2 (0.6%) |
| HLGT: Male reproductive tract infections and | 0 | 1 (0.3%) |
| inflammations | | |
| HLT: Prostate and seminal vesicles infections and | 0 | 1 (0.3%) |
| inflammations | | |
| Prostatitis | 0 | 1 (0.3%) |
| HLGT: Prostatic disorders (excl infections and | 0 | 1 (0.3%) |
| inflammations) | | |
| HLT: Prostatic neoplasms and hypertrophy | 0 | 1 (0.3%) |
| Benign prostatic hyperplasia | 0 | 1 (0.3%) |
| Any class | 26 (8.2%) | 22 (7.0%) |
| GENERAL DISORDERS AND ADMINISTRATION SITE | 2 (0.6%) | 2 (0.6%) |
| CONDITIONS | | |
| HLGT: Body temperature conditions | 1 (0.3%) | 0 |
| HLT: Febrile disorders | 1 (0.3%) | 0 |
| Pyrexia | 1 (0.3%) | 0 |
| HLGT: General system disorders NEC | 1 (0.3%) | 2 (0.6%) |
| HLT: Pain and discomfort NEC | 1 (0.3%) | 2 (0.6%) |
| Non-cardiac chest pain | 1 (0.3%) | 1 (0.3%) |
| Pain | 0 | 1 (0.3%) |
| INJURY, POISONING AND PROCEDURAL | 1 (0.3%) | 1 (0.3%) |
| COMPLICATIONS | | |
| HLGT: Injuries NEC | 1 (0.3%) | 0 |
| HLT: Muscle, tendon and ligament injuries | 1 (0.3%) | 0 |
| Tendon rupture | 1 (0.3%) | 0 |
| HLGT: Medication errors | 0 | 1 (0.3%) |
| HLT: Overdoses | 0 | 1 (0.3%) |
| Intentional overdose | 0 | 1 (0.3%) |

TEAE: Treatment Emergent Adverse Event,
SOC: System Organ Class,
HLGT: High Level Group Term,
HLT: High Level term,
PT: Preferred Term.
MedDRA version: 13.1
Note:
Table sorted by SOC internationally agreed order and HLGT, HLT, PT alphabetic order.

TABLE 19
Number (%) of patients experiencing TEAE(s) leading to permanent treatment
discontinuation during the overall treatment period by primary SOC, HLGT, HLT, and PT -
Safety population

| PRIMARY SYSTEM ORGAN CLASS | | |
|---|---|---|
| HLGT: High Level Group Term | | |
| HLT: High Level Term | Lixisenatide | Exenatide |
| Preferred Term | (N = 318) | (N = 316) |
| Any class | 45 (14.2%) | 45 (14.2%) |
| INFECTIONS AND INFESTATIONS | 2 (0.6%) | 0 |
| HLGT: Infections - pathogen unspecified | 2 (0.6%) | 0 |
| HLT: Lower respiratory tract and lung infections | 1 (0.3%) | 0 |
| Bronchitis | 1 (0.3%) | 0 |
| HLT: Sepsis, bacteraemia, viraemia and fungaemia NEC | 1 (0.3%) | 0 |
| Sepsis | 1 (0.3%) | 0 |
| NEOPLASMS BENIGN, MALIGNANT AND UNSPECIFIED | 1 (0.3%) | 1 (0.3%) |
| (INCL CYSTS AND POLYPS) | | |
| HLGT: Gastrointestinal neoplasms malignant and unspecified | 0 | 1 (0.3%) |
| HLT: Pancreatic neoplasms malignant (excl islet cell and | 0 | 1 (0.3%) |
| carcinoid) | | |
| Pancreatic carcinoma | 0 | 1 (0.3%) |
| HLGT: Miscellaneous and site unspecified neoplasms malignant | 1 (0.3%) | 0 |
| and unspecified | | |
| HLT: Neoplasms malignant site unspecified NEC | 1 (0.3%) | 0 |
| Metastatic neoplasm | 1 (0.3%) | 0 |
| BLOOD AND LYMPHATIC SYSTEM DISORDERS | 0 | 1 (0.3%) |
| HLGT: White blood cell disorders | 0 | 1 (0.3%) |
| HLT: Neutropenias | 0 | 1 (0.3%) |
| Neutropenia | 0 | 1 (0.3%) |
| Any class | 45 (14.2%) | 45 (14.2%) |
| IMMUNE SYSTEM DISORDERS | 1 (0.3%) | 0 |
| HLGT: Allergic conditions | 1 (0.3%) | 0 |
| HLT: Allergies to foods, food additives, drugs and other | 1 (0.3%) | 0 |
| chemicals | | |
| Drug hypersensitivity | 1 (0.3%) | 0 |
| METABOLISM AND NUTRITION DISORDERS | 1 (0.3%) | 2 (0.6%) |
| HLGT: Appetite and general nutritional disorders | 0 | 1 (0.3%) |
| HLT: Appetite disorders | 0 | 1 (0.3%) |
| Decreased appetite | 0 | 1 (0.3%) |
| HLGT: Glucose metabolism disorders (incl diabetes mellitus) | 1 (0.3%) | 1 (0.3%) |
| HLT: Hyperglycaemic conditions NEC | 1 (0.3%) | 0 |
| Hyperglycaemia | 1 (0.3%) | 0 |
| HLT: Hypoglycaemic conditions NEC | 0 | 1 (0.3%) |
| Hypoglycaemia | 0 | 1 (0.3%) |
| NERVOUS SYSTEM DISORDERS | 3 (0.9%) | 7 (2.2%) |
| HLGT: Cranial nerve disorders (excl neoplasms) | 0 | 1 (0.3%) |
| HLT: Olfactory nerve disorders | 0 | 1 (0.3%) |
| Hyposmia | 0 | 1 (0.3%) |
| HLGT: Headaches | 2 (0.6%) | 2 (0.6%) |
| HLT: Headaches NEC | 2 (0.6%) | 2 (0.6%) |
| Headache | 2 (0.6%) | 2 (0.6%) |
| HLGT: Mental impairment disorders | 0 | 1 (0.3%) |
| HLT: Mental impairment (excl dementia and memory loss) | 0 | 1 (0.3%) |
| Cognitive disorder | 0 | 1 (0.3%) |
| Any class | 45 (14.2%) | 45 (14.2%) |
| HLGT: Neurological disorders NEC | 0 | 5 (1.6%) |
| HLT: Disturbances in consciousness NEC | 0 | 1 (0.3%) |
| Somnolence | 0 | 1 (0.3%) |
| HLT: Neurological signs and symptoms NEC | 0 | 4 (1.3%) |
| Dizziness | 0 | 4 (1.3%) |
| HLT: Sensory abnormalities NEC | 0 | 1 (0.3%) |
| Hypogeusia | 0 | 1 (0.3%) |
| HLGT: Neuromuscular disorders | 1 (0.3%) | 0 |
| HLT: Muscle tone abnormal | 1 (0.3%) | 0 |
| Hypotonia | 1 (0.3%) | 0 |
| EYE DISORDERS | 0 | 2 (0.6%) |
| HLGT: Eye disorders NEC | 0 | 1 (0.3%) |
| HLT: Ocular disorders NEC | 0 | 1 (0.3%) |
| Eye pain | 0 | 1 (0.3%) |
| HLGT: Vision disorders | 0 | 1 (0.3%) |
| HLT: Visual disorders NEC | 0 | 1 (0.3%) |
| Vision blurred | 0 | 1 (0.3%) |
| EAR AND LABYRINTH DISORDERS | 0 | 2 (0.6%) |
| HLGT: Inner ear and VIIIth cranial nerve disorders | 0 | 2 (0.6%) |
| HLT: Inner ear signs and symptoms | 0 | 2 (0.6%) |
| Motion sickness | 0 | 1 (0.3%) |
| Vertigo | 0 | 1 (0.3%) |
| Any class | 45 (14.2%) | 45 (14.2%) |
| CARDIAC DISORDERS | 2 (0.6%) | 2 (0.6%) |
| HLGT: Cardiac arrhythmias | 1 (0.3%) | 0 |
| HLT: Rate and rhythm disorders NEC | 1 (0.3%) | 0 |
| Arrhythmia | 1 (0.3%) | 0 |
| HLGT: Coronary artery disorders | 1 (0.3%) | 2 (0.6%) |
| HLT: Ischaemic coronary artery disorders | 1 (0.3%) | 2 (0.6%) |
| Acute myocardial infarction | 0 | 1 (0.3%) |
| Myocardial infarction | 0 | 1 (0.3%) |
| Myocardial ischaemia | 1 (0.3%) | 0 |
| GASTROINTESTINAL DISORDERS | 24 (7.5%) | 27 (8.5%) |
| HLGT: Gastrointestinal motility and defaecation conditions | 7 (2.2%) | 7 (2.2%) |
| HLT: Diarrhoea (excl infective) | 6 (1.9%) | 6 (1.9%) |
| Diarrhoea | 6 (1.9%) | 6 (1.9%) |
| HLT: Gastrointestinal atonic and hypomotility disorders NEC | 1 (0.3%) | 0 |
| Constipation | 1 (0.3%) | 0 |
| HLT: Gastrointestinal spastic and hypermotility disorders | 0 | 1 (0.3%) |
| Irritable bowel syndrome | 0 | 1 (0.3%) |
| HLGT: Gastrointestinal signs and symptoms | 19 (6.0%) | 26 (8.2%) |
| HLT: Dyspeptic signs and symptoms | 0 | 1 (0.3%) |
| Dyspepsia | 0 | 1 (0.3%) |
| HLT: Gastrointestinal and abdominal pains (excl oral and | 2 (0.6%) | 4 (1.3%) |
| throat) | | |
| Abdominal pain | 1 (0.3%) | 3 (0.9%) |
| Abdominal pain upper | 1 (0.3%) | 0 |
| Gastrointestinal pain | 0 | 1 (0.3%) |
| Any class | 45 (14.2%) | 45 (14.2%) |
| HLT: Nausea and vomiting symptoms | 18 (5.7%) | 23 (7.3%) |
| Nausea | 15 (4.7%) | 19 (6.0%) |
| Vomiting | 4 (1.3%) | 10 (3.2%) |
| HLGT: Gastrointestinal vascular conditions | 1 (0.3%) | 0 |
| HLT: Haemorrhoids and gastrointestinal varices (excl | 1 (0.3%) | 0 |
| oesophageal) | | |
| Haemorrhoids | 1 (0.3%) | 0 |
| HEPATOBILIARY DISORDERS | 0 | 1 (0.3%) |
| HLGT: Gallbladder disorders | 0 | 1 (0.3%) |
| HLT: Cholecystitis and cholelithiasis | 0 | 1 (0.3%) |
| Cholelithiasis | 0 | 1 (0.3%) |
| SKIN AND SUBCUTANEOUS TISSUE DISORDERS | 1 (0.3%) | 1 (0.3%) |
| HLGT: Epidermal and dermal conditions | 1 (0.3%) | 0 |
| HLT: Dermatitis and eczema | 1 (0.3%) | 0 |
| Dermatitis | 1 (0.3%) | 0 |
| HLGT: Skin appendage conditions | 0 | 1 (0.3%) |
| HLT: Apocrine and eccrine gland disorders | 0 | 1 (0.3%) |
| Hyperhidrosis | 0 | 1 (0.3%) |
| MUSCULOSKELETAL AND CONNECTIVE TISSUE | 1 (0.3%) | 0 |
| DISORDERS | | |
| HLGT: Joint disorders | 1 (0.3%) | 0 |
| HLT: Rheumatoid arthropathies | 1 (0.3%) | 0 |
| Rheumatoid arthritis | 1 (0.3%) | 0 |
| Any class | 45 (14.2%) | 45 (14.2%) |
| PREGNANCY, PUERPERIUM AND PERINATAL CONDITIONS | 0 | 1 (0.3%) |
| HLGT: Pregnancy, labour, delivery and postpartum conditions | 0 | 1 (0.3%) |
| HLT: Normal pregnancy, labour and delivery | 0 | 1 (0.3%) |
| Pregnancy | 0 | 1 (0.3%) |
| REPRODUCTIVE SYSTEM AND BREAST DISORDERS | 0 | 1 (0.3%) |
| HLGT: Sexual function and fertility disorders | 0 | 1 (0.3%) |
| HLT: Erection and ejaculation conditions and disorders | 0 | 1 (0.3%) |
| Erectile dysfunction | 0 | 1 (0.3%) |
| GENERAL DISORDERS AND ADMINISTRATION SITE | 5 (1.6%) | 3 (0.9%) |
| CONDITIONS | | |
| HLGT: Administration site reactions | 3 (0.9%) | 0 |
| HLT: Injection site reactions | 3 (0.9%) | 0 |
| Injection site hypersensitivity | 1 (0.3%) | 0 |
| Injection site pain | 1 (0.3%) | 0 |
| Injection site reaction | 1 (0.3%) | 0 |
| HLGT: Body temperature conditions | 1 (0.3%) | 0 |
| HLT: Febrile disorders | 1 (0.3%) | 0 |
| Pyrexia | 1 (0.3%) | 0 |
| HLGT: General system disorders NEC | 1 (0.3%) | 3 (0.9%) |
| HLT: Asthenic conditions | 1 (0.3%) | 3 (0.9%) |
| Fatigue | 0 | 2 (0.6%) |
| Malaise | 1 (0.3%) | 1 (0.3%) |
| Any class | 45 (14.2%) | 45 (14.2%) |
| INVESTIGATIONS | 6 (1.9%) | 4 (1.3%) |
| HLGT: Endocrine investigations (incl sex hormones) | 3 (0.9%) | 0 |
| HLT: Gastrointestinal, pancreatic and APUD hormone | 3 (0.9%) | 0 |
| analyses | | |
| Blood calcitonin increased | 3 (0.9%) | 0 |
| HLGT: Gastrointestinal investigations | 2 (0.6%) | 2 (0.6%) |
| HLT: Digestive enzymes | 2 (0.6%) | 2 (0.6%) |
| Blood amylase increased | 0 | 1 (0.3%) |
| Lipase increased | 1 (0.3%) | 1 (0.3%) |
| Pancreatic enzymes increased | 1 (0.3%) | 1 (0.3%) |
| HLGT: Hepatobiliary investigations | 0 | 1 (0.3%) |
| HLT: Liver function analyses | 0 | 1 (0.3%) |
| Liver function test abnormal | 0 | 1 (0.3%) |
| HLGT: Physical examination topics | 1 (0.3%) | 1 (0.3%) |
| HLT: Physical examination procedures | 1 (0.3%) | 1 (0.3%) |
| Weight decreased | 1 (0.3%) | 1 (0.3%) |

TEAE: Treatment Emergent Adverse Event,
SOC: System Organ Class,
HLGT: High Level Group Term,
HLT: High Level term,
PT: Preferred Term.
MedDRA version: 13.1
Note:
Table sorted by SOC internationally agreed order and HLGT, HLT, PT alphabetic order.

### Hypoglycemia

Sixteen (5.0%) lixisenatide-treated patients had symptomatic hypoglycemia events per protocol definition during the on-treatment period for the whole study, whereas 46 (14.6%) exenatide-treated patients reported symptomatic hypoglycemia during the same period (Table 20). None of the symptomatic hypoglycemia events was severe in intensity.

### Symptomatic Hypoglycemia

Symptomatic hypoglycemia is defined as an event with clinical symptoms that are considered to result from a hypoglycemic episode (e.g., sweating, palpitations, hunger, restlessness, anxiety, fatigue, irritability, headache, loss of concentration, somnolence, psychiatric or visual disorders, transient sensory or motor defects, confusion, convulsions, or coma) with an accompanying plasma glucose <60 mg/dL (3.3 mmol/L) or associated with prompt recovery after oral carbohydrate administration if no plasma glucose value is available. Symptoms with an associated plasma glucose ≧60 mg/dL (3.3 mmol/L) should not be reported as a hypoglycemia.

Symptomatic hypoglycemia is to be reported as an adverse event. Additional information should be collected on a specific symptomatic hypoglycemic event complementary form.

### Severe Symptomatic Hypoglycemia

Severe symptomatic hypoglycemia is defined as an event with clinical symptoms that are considered to result from hypoglycemia in which the patient required the assistance of another person, because the patient could not treat him/herself due to acute neurological impairment directly resulting from the hypoglycemic event, and one of the following:
(f) The event was associated with a plasma glucose level below 36 mg/dL (2.0 mmol/L).
(g) If no plasma glucose value is available, then the event was associated with prompt recovery after oral carbohydrate, intravenous glucose, or glucagon administration.

The definition of severe symptomatic hypoglycemia includes all episodes in which neurological impairment was severe enough to prevent self-treatment and which were thus thought to place patients at risk for injury to themselves or others. Note that “requires assistance” means that the patient could not help himself or herself. Someone being kind that assists spontaneously the patient when not necessary does not qualify as “requires assistance.”

Severe symptomatic hypoglycemia will be qualified as an SAE only if it fulfills SAE criteria.

TABLE 20
Summary of symptomatic hypoglycemia during the on-treatment period
for the whole study - Safety population

| | Lixisenatide | Exenatide |
|---|---|---|
| Type | (N = 318) | (N = 316) |
| Total patient years | 431.47 | 420.63 |
| Any symptomatic hypoglycemia | | |
| Number of patients with events, n (%)^{a} | 16 (5.0%) | 46 (14.6%) |
| Number of patients with events per | 3.7 | 10.9 |
| 100 patient years^{b} | | |
| Blood glucose <60 mg/dL | | |
| Number of patients with events, n (%)^{a} | 15 (4.7%) | 38 (12.0%) |
| Number of patients with events per | 3.5 | 9 |
| 100 patient years^{b} | | |
| No blood glucose reported | | |
| Number of patients with events, n (%)^{a} | 3 (0.9%) | 11 (3.5%) |
| Number of patients with events per | 0.7 | 2.6 |
| 100 patient years^{b} | | |

^{a}Percents are calculated using the number of safety patients as the denominator.
^{b}Calculated as (number of patients with events * 100 divided by total exposure + 3 days in patient years).
Note:
Symptomatic hypoglycemia = symptomatic hypoglycemia as defined per protocol.

Thirty-six patients (9.1% for lixisenatide and 2.2% for exenatide) experienced injection site reaction AEs (Table 21). The injection site reaction AEs were identified by searching the term “injection site” in either the investigator reported AE PTs or PTs from the ARAC diagnosis during the allergic reaction adjudication. None of the reactions was serious or severe.

TABLE 21
Number (%) of patients experiencing injection site reactions
during the overall treatment period - Safety population

| Event source | Lixisenatide | Exenatide |
|---|---|---|
| Preferred Term | (N = 318) | (N = 316) |
| Any injection site reactions | 29 (9.1%) | 7 (2.2%) |
| Investigator reported PTs | 25 (7.9%) | 7 (2.2%) |
| Injection site erythema | 5 (1.6%) | 0 |
| Injection site pain | 5 (1.6%) | 0 |
| Injection site pruritus | 5 (1.6%) | 0 |
| Injection site reaction | 5 (1.6%) | 2 (0.6%) |
| Injection site haematoma | 3 (0.9%) | 3 (0.9%) |
| Injection site rash | 3 (0.9%) | 1 (0.3%) |
| Injection site discomfort | 1 (0.3%) | 0 |
| Injection site hypersensitivity | 1 (0.3%) | 0 |
| Injection site inflammation | 1 (0.3%) | 0 |
| Injection site urticaria | 1 (0.3%) | 0 |
| Injection site haemorrhage | 0 | 1 (0.3%) |
| PTs by ARAC diagnosis | 9 (2.8%) | 1 (0.3%) |
| Injection site reaction | 8 (2.5%) | 1 (0.3%) |
| Injection site urticaria | 1 (0.3%) | 0 |

ARAC = Allergic Reaction Assessment Committee.

A total of 42 cases were reported as a suspected allergic event by investigators during the on-treatment period of the whole study and sent to ARAC for adjudication. Thirteen of them (in 6 (1.9%) lixisenatide-treated patients and 3 (0.9%) exenatide-treated patients) were adjudicated as an allergic reaction by the ARAC, but none of the events was adjudicated as possibly related to IP.

TABLE 22
Number (%) of patients with events adjudicated as allergic reaction by ARAC during
the on-treatment period of the whole study - Safety population

| Relationship to | MedDRA coded term (PT) | ARAC | Lixisenatide | Exenatide |
|---|---|---|---|---|
| study treatment | for ARAC diagnosis | diagnosis | (N = 318) | (N = 316) |
| All | Events adjudicated | | 6 (1.9%) | 3 (0.9%) |
| | as an allergic | | | |
| | reaction by ARAC | | | |
| | Allergy to arthropod sting | REACTION | 1 (0.3%) | 0 |
| | | TO WASP | | |
| | Angioedema | ANGIOEDEMA | 0 | 1 (0.3%) |
| | Erythema multiforme | ERYTHEMA | 1 (0.3%) | 0 |
| | | MULTIFORME | | |
| | Rash | FACIAL RASH | 0 | 1 (0.3%) |
| | Rhinitis allergic | ALLERGIC | 2 (0.6%) | 1 (0.3%) |
| | | RHINITIS | | |
| | Urticaria | URTICARIA | 2 (0.6%) | 0 |
| | | (HIVES) | | |
| Possibly Related to IP | Events adjudicated | | 0 | 0 |
| | as an allergic | | | |
| | reaction by ARAC | | | |
| Not related to IP | Events adjudicated | | 6 (1.9%) | 3 (0.9%) |
| | as an allergic | | | |
| | reaction by ARAC | | | |
| | Allergy to arthropod sting | REACTION | 1 (0.3%) | 0 |
| | | TO WASP | | |
| | Angioedema | ANGIOEDEMA | 0 | 1 (0.3%) |
| All | Events adjudicated | | 6 (1.9%) | 3 (0.9%) |
| | as an allergic | | | |
| | reaction by ARAC | | | |
| | Erythema multiforme | ERYTHEMA | 1 (0.3%) | 0 |
| | | MULTIFORME | | |
| | Rash | FACIAL RASH | 0 | 1 (0.3%) |
| | Rhinitis allergic | ALLERGIC | 2 (0.6%) | 1 (0.3%) |
| | | RHINITIS | | |
| | Urticaria | URTICARIA | 2 (0.6%) | 0 |
| | | (HIVES) | | |

ARAC = Allergic Reaction Assessment Committee.
IP = Investigational product.

During the on-treatment period of the whole study, 5 (1.6%) lixisenatide-treated patients and 9 (2.8%) exenatide-treated patients reported events of changes in pancreatic enzymes or lipase or amylase on a specific AE page for “suspected pancreatitis” following the protocol recommendation (Table 23). Patients with at least one value of lipase or amylase 3 ULN are summarized in Table 24. One lixisenatide-treated patient, who reported one increase of lipase and one pancreatic enzymes increased event on the specific AE page, had a lipase value ≧3ULN as well as amylase value >3ULN during the treatment period. No case of acute pancreatitis was observed in the study.

The same number of patients (11 [3.5%] patients in lixisenatide and 11 [3.6%] in exenatide) with elevated lipase (≧3ULN) was observed in each treatment group [Table 24]. Three (1.0%) patients in the lixisenatide group had elevated amylase (≧3ULN), and none in the exenatide group.

TABLE 23
Number (%) of patients with suspected pancreatitis during the
on-treatment period for the whole study - Safety population

| | Lixisenatide | Exenatide |
|---|---|---|
| Preferred Term | (N = 318) | (N = 316) |
| Any | 5 (1.6%) | 9 (2.8%) |
| Blood amylase increased | 2 (0.6%) | 1 (0.3%) |
| Lipase increased | 3 (0.9%) | 7 (2.2%) |
| Pancreatic enzymes increased | 1 (0.3%) | 2 (0.6%) |

n (%) = number and percentage of patients with any cases reported on the AE form for suspected pancreatitis.

TABLE 24
Pancreatic enzymes: Number (%) of patients with at least one
post-baseline PCSA during the on-treatment period for the
whole study according to baseline status - Safety population

| Laboratory criteria | | |
|---|---|---|
| Baseline | Lixisenatide | Exenatide |
| By PCSA criteria n/N1 (%) | (N = 318) | (N = 316) |
| Lipase (IU/L) | | |
| Total* | | |
| ≧3 ULN | 11/311 (3.5%) | 11/306 (3.6%) |
| Normal/Missing | | |
| ≧3 ULN | 10/307 (3.3%) | 10/304 (3.3%) |
| Amylase (IU/L) | | |
| Total* | | |
| ≧3 ULN | 3/311 (1.0%) | 0/306 |
| Lipase (IU/L) | | |
| Normal/Missing | | |
| ≧3 ULN | 3/311 (1.0%) | 0/306 |

PCSA: Potentially Clinically Significant Abnormalities,
ULN = Upper limit of normal.
*Regardless of baseline.
Note:
The number (n) represents the subset of the total number of patients who met the criterion in question at least once. The denominator (/N1) for each parameter within a treatment group is the number of patients for the treatment group who had that parameter assessed post-baseline by baseline PCSA status. Only the worsening of the worst case for each patient is presented by baseline status.

Eight patients (4 [1.3%] in each group) reported a calcitonin value ≧20 ng/L on a specific AE page for “increased calcitonin” (Table 25). No value ≧50 ng/L was reported.

Five (1.8%) patients in the lixisenatide group and 8 (3.0%) patients in the exenatide group had a value of calcitonin ≧20 ng/L during the on treatment period (Table 26). It should be pointed out that calcitonin measurements were added in a protocol amendment after all patients were already randomized. Therefore baseline values are missing for all patients.

TABLE 25
Number (%) of patients with increased calcitonin during the
on-treatment period for the whole study - Safety population

| | Lixisenatide | Exenatide |
|---|---|---|
| Preferred Term | (N = 318) | (N = 316) |
| Any | 4 (1.3%) | 4 (1.3%) |
| Blood calcitonin increased | 4 (1.3%) | 4 (1.3%) |

n (%) = number and percentage of patients with any cases reported on the AE form for increased calcitonin ≧20 ng/L.

TABLE 26
Serum calcitonin - Number (%) of patients by pre-defined categories
during the on-treatment period of the whole study according to
baseline category - Safety population

| Laboratory criteria | | |
|---|---|---|
| Baseline status | Lixisenatide | Exenatide |
| Post-baseline | (N = 318) | (N = 316) |
| Calcitonin (ng/L) | | |
| Total* | | |
| ≦ULN | 244/273 (89.4%) | 232/265 (87.5%) |
| >ULN-<20 ng/L | 24/273 (8.8%) | 25/265 (9.4%) |
| ≧20 ng/L-<50 ng/L | 5/273 (1.8%) | 8/265 (3.0%) |
| ≧50 ng/L | 0/273 | 0/265 |
| Missing | | |
| ≦ULN | 244/273 (89.4%) | 232/265 (87.5%) |
| >ULN-<20 ng/L | 24/273 (8.8%) | 25/265 (9.4%) |
| ≧20 ng/L-<50 ng/L | 5/273 (1.8%) | 8/265 (3.0%) |
| ≧50 ng/L | 0/273 | 0/265 |

ULN = Upper limit of normal
*Regardless of baseline.
Note:
The numerator represents the number of patients who were in the pre-specified categories in each baseline category.
The denominator for each parameter within a treatment group is the number of patients for the treatment group who had that parameter assessed post-baseline by baseline status.
A patient is counted only in the worst category.

Health Related Quality-of-Life (PAGI-QOL Questionnaire)

Table 27 summarizes the ANCOVA analysis result of PAGI-QOL total score. The LS mean changes in PAGI-QOL total score from baseline to Week 24 was −0.09 for the lixisenatide group and −0.06 for the exenatide group (LS mean difference versus exenatide=−0.03).

TABLE 27
Mean change in PAGI-QOL total score
from baseline to week 24 - mITT population

| | Lixisenatide | Exenatide |
|---|---|---|
| PAGI-QOL total score | (N = 311) | (N = 305) |
| Baseline | | |
| Number | 302 | 292 |
| Mean (SD) | 0.60 (0.72) | 0.56 (0.73) |
| Median | 0.27 | 0.27 |
| Min:Max | 0.0:3.1 | 0.0:3.5 |
| Week 24 (LOCF) | | |
| Number | 302 | 292 |
| Mean (SD) | 0.49 (0.64) | 0.50 (0.67) |
| Median | 0.19 | 0.29 |
| Min:Max | 0.0:3.0 | 0.0:3.9 |
| Change from baseline to week 24 | | |
| (LOCF) | | |
| Number | 302 | 292 |
| Mean (SD) | −0.11 (0.52) | −0.06 (0.57) |
| Median | 0.00 | 0.00 |
| Min:Max | −2.1:1.5 | −1.9:3.5 |
| LS Mean (SE)^{a} | −0.09 (0.031) | −0.06 (0.032) |
| LS Mean difference (SE) vs. | −0.03 (0.039) | |
| Exenatide^{a} | | |
| 95% CI | (−0.111 to 0.043) | |

LOCF = Last observation carry forward.
^{a}Analysis of covariance (ANCOVA) model with treatment groups (Exenatide and Lixisenatide), randomization strata of screening HbA1c (<8.0, ≧8.0%), randomization strata of screening BMI (<30, ≧30 kg/m²), and country as fixed effects and baseline PAGI-QOL total score as a covariate.
Note:
The analysis included measurements obtained before the introduction of rescue medication and up to 3 days after the last dose of the investigational product injection on or before Visit 11 (Week24), or Day 169 if Visit 11 (Week 24) is not available. Patients with both baseline and Week 24 (LOCF) measurements are included.

### APPENDIX

TABLE 28
Mean change in HbA1c (%) from baseline by visit-mITT population

| Treatment | Observed data | | | | | | | Chang [symbol] | | |
|---|---|---|---|---|---|---|---|---|---|---|
| Time point | N | Mean | SD | SE | Median | Min | Max | N | Mean | SD |
| Lixisenatide (N = 311) | | | | | | | | | | |
| Screening | 311 | 8.04 | 0.80 | 0.046 | 7.90 | 7.0 | 10.0 | | | |
| Baseline | 311 | 7.97 | 0.81 | 0.046 | 7.80 | 6.1 | 10.2 | | | |
| Week 8 | 285 | 7.34 | 0.81 | 0.048 | 7.20 | 5.4 | 11.2 | 285 | −0.61 | 0.61 |
| Week 12 | 282 | 7.21 | 0.88 | 0.052 | 7.00 | 5.8 | 11.6 | 282 | −0.75 | 0.74 |
| Week 24 | 266 | 7.10 | 0.91 | 0.056 | 6.90 | 5.3 | 11.1 | 266 | −0.86 | 0.88 |
| Week 24 (LOCF) | 295 | 7.17 | 0.96 | 0.056 | 7.00 | 5.3 | 11.1 | 295 | −0.80 | 0.88 |
| Week 36 | 238 | 7.09 | 0.86 | 0.056 | 6.90 | 5.6 | 10.2 | 238 | −0.83 | 0.80 |
| Week 44 | 218 | 7.08 | 0.89 | 0.061 | 6.90 | 5.5 | 11.9 | 218 | −0.81 | 0.86 |
| Week 52 | 212 | 7.03 | 0.87 | 0.060 | 6.90 | 5.5 | 11.4 | 212 | −0.85 | 0.85 |
| Week 60 | 194 | 7.03 | 0.91 | 0.065 | 6.90 | 5.4 | 11.2 | 194 | −0.82 | 0.89 |
| Week 68 | 190 | 6.98 | 0.87 | 0.063 | 6.90 | 5.4 | 10.8 | 190 | −0.85 | 0.87 |
| Week 76 | 180 | 6.97 | 0.85 | 0.063 | 6.90 | 5.3 | 10.7 | 180 | −0.86 | 0.90 |
| Week 84 | 110 | 7.04 | 0.89 | 0.085 | 6.80 | 5.6 | 9.6 | 110 | −0.86 | 0.99 |
| Week 92 | 52 | 7.03 | 0.89 | 0.123 | 6.85 | 5.6 | 10.7 | 52 | −0.82 | 1.08 |
| Week 100 | 25 | 7.10 | 0.72 | 0.144 | 7.00 | 6.0 | 9.3 | 25 | −0.76 | 0.94 |
| Week 108 | 5 | 7.44 | 1.12 | 0.503 | 7.00 | 6.6 | 9.4 | 5 | −0.62 | 1.01 |
| Last value on- | 295 | 7.44 | 1.12 | 0.065 | 7.20 | 4.4 | 11.1 | 295 | −0.53 | 1.02 |
| treatment (LOCF) | | | | | | | | | | |
| Exenatide (N = 305) | | | | | | | | | | |
| Screening | 305 | 8.02 | 0.78 | 0.045 | 7.90 | 7.0 | 10.0 | | | |
| Baseline | 305 | 7.96 | 0.78 | 0.045 | 7.90 | 6.1 | 9.9 | | | |
| Week 8 | 286 | 7.15 | 0.78 | 0.046 | 7.00 | 5.5 | 10.1 | 286 | −0.82 | 0.67 |
| Week 12 | 274 | 7.00 | 0.83 | 0.050 | 6.90 | 5.2 | 10.1 | 274 | −0.98 | 0.75 |
| Week 24 | 258 | 6.94 | 0.87 | 0.054 | 6.90 | 4.9 | 10.7 | 258 | −1.03 | 0.87 |
| Week 24 (LOCF) | 297 | 7.01 | 0.88 | 0.051 | 7.00 | 4.9 | 10.7 | 297 | −0.95 | 0.87 |
| Week 36 | 223 | 6.82 | 0.75 | 0.051 | 6.70 | 5.2 | 9.8 | 223 | −1.14 | 0.84 |
| Week 44 | 212 | 6.77 | 0.73 | 0.050 | 6.70 | 5.3 | 9.5 | 212 | −1.16 | 0.85 |
| Week 52 | 204 | 6.77 | 0.76 | 0.053 | 6.70 | 5.1 | 9.3 | 204 | −1.16 | 0.86 |
| Week 60 | 193 | 6.77 | 0.80 | 0.057 | 6.70 | 5.0 | 9.7 | 193 | −1.14 | 0.90 |
| Week 68 | 186 | 6.67 | 0.78 | 0.057 | 6.60 | 4.8 | 9.7 | 186 | −1.21 | 0.92 |
| Week 76 | 176 | 6.71 | 0.80 | 0.061 | 6.60 | 5.0 | 9.6 | 176 | −1.19 | 0.90 |
| Week 84 | 109 | 6.75 | 0.80 | 0.077 | 6.60 | 5.1 | 10.0 | 109 | −1.19 | 1.00 |
| Week 92 | 51 | 6.85 | 0.94 | 0.132 | 6.50 | 5.7 | 10.2 | 51 | −1.07 | 1.01 |
| Week 100 | 27 | 6.90 | 1.15 | 0.222 | 6.60 | 5.7 | 10.6 | 27 | −1.04 | 1.06 |
| Lixisenatide (N = 311) | | | | | | | | | | |
| Week 108 | 3 | 7.50 | 1.68 | 0.971 | 6.90 | 6.2 | 9.4 | 3 | −1.17 | 1.71 |
| Week 116 | 1 | 6.80 | NC | NC | 6.80 | 6.8 | 6.8 | 1 | −2.40 | NC |
| Last value on- | 297 | 7.09 | 1.01 | 0.058 | 6.90 | 4.9 | 11.8 | 297 | −0.87 | 1.02 |
| treatment (LOCF) | | | | | | | | | | |

LOCF = Last observation carry forward.
Note:
The analysis excluded measurements obtained after the introduction of rescue medication and/or after the treatment cessation p [symbol] For Week 24 (LOCF), the analysis included measurements obtained up to 3 days after the last dose of the investigational product inje [symbol] Day 169 if Visit 11 (Week 24) is not available. [symbol]
indicates data missing or illegible when filed

TABLE 29
Number (%) of patients experiencing common TEAE(s) (PT ≧1% in any treatment
group) by primary SOC, HLGT, HLT and PT during the on-treatment period for the whole study -
Safety population

| Primary System Organ Class | | | | |
|---|---|---|---|---|
| HLGT: High Level Group Term | | | | |
| HLT: High Level Term | Lixisenatide | | Exenatide | |
| Preferred Term | (N = 318) | | (N = 316) | |
| Any class | 257 | (80.8%) | 264 | (83.5%) |
| INFECTIONS AND INFESTATIONS | 140 | (44.0%) | 126 | (39.9%) |
| HLGT: Fungal infectious disorders | 10 | (3.1%) | 5 | (1.6%) |
| HLT: Fungal infections NEC | 8 | (2.5%) | 3 | (0.9%) |
| Fungal infection | 4 | (1.3%) | 1 | (0.3%) |
| HLGT: Infections - pathogen unspecified | 125 | (39.3%) | 104 | (32.9%) |
| HLT: Abdominal and gastrointestinal infections | 12 | (3.8%) | 16 | (5.1%) |
| Gastroenteritis | 11 | (3.5%) | 14 | (4.4%) |
| HLT: Dental and oral soft tissue infections | 10 | (3.1%) | 3 | (0.9%) |
| Tooth infection | 5 | (1.6%) | 1 | (0.3%) |
| HLT: Lower respiratory tract and lung infections | 25 | (7.9%) | 25 | (7.9%) |
| Bronchitis | 20 | (6.3%) | 19 | (6.0%) |
| Pneumonia | 5 | (1.6%) | 5 | (1.6%) |
| Any class | 257 | (80.8%) | 264 | (83.5%) |
| HLT: Upper respiratory tract infections | 84 | (26.4%) | 68 | (21.5%) |
| Nasopharyngitis | 49 | (15.4%) | 35 | (11.1%) |
| Pharyngitis | 8 | (2.5%) | 7 | (2.2%) |
| Rhinitis | 6 | (1.9%) | 6 | (1.9%) |
| Sinusitis | 10 | (3.1%) | 10 | (3.2%) |
| Upper respiratory tract infection | 18 | (5.7%) | 13 | (4.1%) |
| HLT: Urinary tract infections | 19 | (6.0%) | 17 | (5.4%) |
| Urinary tract infection | 14 | (4.4%) | 15 | (4.7%) |
| HLGT: Viral infectious disorders | 40 | (12.6%) | 39 | (12.3%) |
| HLT: Influenza viral infections | 29 | (9.1%) | 32 | (10.1%) |
| Influenza | 29 | (9.1%) | 32 | (10.1%) |
| HLT: Viral infections NEC | 8 | (2.5%) | 6 | (1.9%) |
| Gastroenteritis viral | 3 | (0.9%) | 4 | (1.3%) |
| Viral infection | 4 | (1.3%) | 2 | (0.6%) |
| METABOLISM AND NUTRITION DISORDERS | 44 | (13.8%) | 65 | (20.6%) |
| HLGT: Appetite and general nutritional disorders | 9 | (2.8%) | 12 | (3.8%) |
| HLT: Appetite disorders | 9 | (2.8%) | 12 | (3.8%) |
| Decreased appetite | 6 | (1.9%) | 11 | (3.5%) |
| HLGT: Glucose metabolism disorders (incl diabetes mellitus) | 25 | (7.9%) | 50 | (15.8%) |
| HLT: Hyperglycaemic conditions NEC | 6 | (1.9%) | 2 | (0.6%) |
| Hyperglycaemia | 6 | (1.9%) | 2 | (0.6%) |
| HLT: Hypoglycaemic conditions NEC | 20 | (6.3%) | 49 | (15.5%) |
| Hypoglycaemia | 18 | (5.7%) | 48 | (15.2%) |
| PSYCHIATRIC DISORDERS | 38 | (11.9%) | 17 | (5.4%) |
| HLGT: Anxiety disorders and symptoms | 16 | (5.0%) | 9 | (2.8%) |
| HLT: Anxiety symptoms | 14 | (4.4%) | 8 | (2.5%) |
| Anxiety | 10 | (3.1%) | 5 | (1.6%) |
| Any class | 257 | (80.8%) | 264 | (83.5%) |
| HLGT: Depressed mood disorders and disturbances | 13 | (4.1%) | 5 | (1.6%) |
| HLT: Depressive disorders | 13 | (4.1%) | 5 | (1.6%) |
| Depression | 13 | (4.1%) | 5 | (1.6%) |
| HLGT: Sleep disorders and disturbances | 8 | (2.5%) | 7 | (2.2%) |
| HLT: Disturbances in initiating and maintaining sleep | 6 | (1.9%) | 7 | (2.2%) |
| Insomnia | 5 | (1.6%) | 7 | (2.2%) |
| NERVOUS SYSTEM DISORDERS | 76 | (23.9%) | 69 | (21.8%) |
| HLGT: Headaches | 48 | (15.1%) | 31 | (9.8%) |
| HLT: Headaches NEC | 46 | (14.5%) | 31 | (9.8%) |
| Headache | 46 | (14.5%) | 31 | (9.8%) |
| HLGT: Movement disorders (incl parkinsonism) | 4 | (1.3%) | 8 | (2.5%) |
| HLT: Tremor (excl congenital) | 4 | (1.3%) | 8 | (2.5%) |
| Tremor | 4 | (1.3%) | 8 | (2.5%) |
| HLGT: Neurological disorders NEC | 29 | (9.1%) | 42 | (13.3%) |
| HLT: Neurological signs and symptoms NEC | 21 | (6.6%) | 31 | (9.8%) |
| Dizziness | 21 | (6.6%) | 31 | (9.8%) |
| HLGT: Peripheral neuropathies | 5 | (1.6%) | 6 | (1.9%) |
| HLT: Chronic polyneuropathies | 4 | (1.3%) | 3 | (0.9%) |
| Diabetic neuropathy | 4 | (1.3%) | 3 | (0.9%) |
| HLGT: Spinal cord and nerve root disorders | 5 | (1.6%) | 1 | (0.3%) |
| HLT: Lumbar spinal cord and nerve root disorders | 4 | (1.3%) | 1 | (0.3%) |
| Sciatica | 4 | (1.3%) | 1 | (0.3%) |
| EYE DISORDERS | 21 | (6.6%) | 18 | (5.7%) |
| HLGT: Vision disorders | 5 | (1.6%) | 6 | (1.9%) |
| HLT: Visual disorders NEC | 4 | (1.3%) | 5 | (1.6%) |
| Vision blurred | 3 | (0.9%) | 5 | (1.6%) |
| Any class | 257 | (80.8%) | 264 | (83.5%) |
| EAR AND LABYRINTH DISORDERS | 11 | (3.5%) | 13 | (4.1%) |
| HLGT: Inner ear and VIIIth cranial nerve disorders | 8 | (2.5%) | 9 | (2.8%) |
| HLT: Inner ear signs and symptoms | 8 | (2.5%) | 9 | (2.8%) |
| Vertigo | 5 | (1.6%) | 6 | (1.9%) |
| CARDIAC DISORDERS | 16 | (5.0%) | 12 | (3.8%) |
| HLGT: Cardiac arrhythmias | 12 | (3.8%) | 6 | (1.9%) |
| HLT: Rate and rhythm disorders NEC | 5 | (1.6%) | 3 | (0.9%) |
| Tachycardia | 4 | (1.3%) | 2 | (0.6%) |
| HLT: Supraventricular arrhythmias | 5 | (1.6%) | 1 | (0.3%) |
| Atrial fibrillation | 4 | (1.3%) | 1 | (0.3%) |
| VASCULAR DISORDERS | 25 | (7.9%) | 18 | (5.7%) |
| HLGT: Decreased and nonspecific blood pressure disorders and | 2 | (0.6%) | 4 | (1.3%) |
| shock | | | | |
| HLT: Vascular hypotensive disorders | 2 | (0.6%) | 4 | (1.3%) |
| Hypotension | 2 | (0.6%) | 4 | (1.3%) |
| HLGT: Vascular hypertensive disorders | 19 | (6.0%) | 11 | (3.5%) |
| HLT: Vascular hypertensive disorders NEC | 19 | (6.0%) | 10 | (3.2%) |
| Hypertension | 19 | (6.0%) | 10 | (3.2%) |
| RESPIRATORY, THORACIC AND MEDIASTINAL DISORDERS | 34 | (10.7%) | 29 | (9.2%) |
| HLGT: Respiratory disorders NEC | 23 | (7.2%) | 19 | (6.0%) |
| HLT: Coughing and associated symptoms | 10 | (3.1%) | 13 | (4.1%) |
| Cough | 10 | (3.1%) | 12 | (3.8%) |
| HLT: Upper respiratory tract signs and symptoms | 10 | (3.1%) | 6 | (1.9%) |
| Oropharyngeal pain | 7 | (2.2%) | 5 | (1.6%) |
| Any class | 257 | (80.8%) | 264 | (83.5%) |
| GASTROINTESTINAL DISORDERS | 163 | (51.3%) | 177 | (56.0%) |
| HLGT: Gastrointestinal inflammatory conditions | 10 | (3.1%) | 10 | (3.2%) |
| HLT: Gastritis (excl infective) | 9 | (2.8%) | 7 | (2.2%) |
| Gastritis | 9 | (2.8%) | 7 | (2.2%) |
| HLGT: Gastrointestinal motility and defaecation conditions | 61 | (19.2%) | 68 | (21.5%) |
| HLT: Diarrhoea (excl infective) | 48 | (15.1%) | 54 | (17.1%) |
| Diarrhoea | 48 | (15.1%) | 54 | (17.1%) |
| HLT: Gastrointestinal atonic and hypomotility disorders NEC | 19 | (6.0%) | 23 | (7.3%) |
| Constipation | 14 | (4.4%) | 17 | (5.4%) |
| Gastrooesophageal reflux disease | 5 | (1.6%) | 6 | (1.9%) |
| HLGT: Gastrointestinal signs and symptoms | 132 | (41.5%) | 151 | (47.8%) |
| HLT: Dyspeptic signs and symptoms | 22 | (6.9%) | 24 | (7.6%) |
| Dyspepsia | 19 | (6.0%) | 21 | (6.6%) |
| HLT: Flatulence, bloating and distension | 15 | (4.7%) | 17 | (5.4%) |
| Abdominal distension | 7 | (2.2%) | 8 | (2.5%) |
| Flatulence | 9 | (2.8%) | 12 | (3.8%) |
| HLT: Gastrointestinal and abdominal pains (excl oral and | 27 | (8.5%) | 22 | (7.0%) |
| throat) | | | | |
| Abdominal pain | 14 | (4.4%) | 8 | (2.5%) |
| Abdominal pain upper | 16 | (5.0%) | 14 | (4.4%) |
| HLT: Gastrointestinal signs and symptoms NEC | 5 | (1.6%) | 7 | (2.2%) |
| Abdominal discomfort | 4 | (1.3%) | 6 | (1.9%) |
| HLT: Nausea and vomiting symptoms | 104 | (32.7%) | 128 | (40.5%) |
| Nausea | 91 | (28.6%) | 119 | (37.7%) |
| Vomiting | 41 | (12.9%) | 49 | (15.5%) |
| Any class | 257 | (80.8%) | 264 | (83.5%) |
| HEPATOBILIARY DISORDERS | 11 | (3.5%) | 8 | (2.5%) |
| HLGT: Hepatic and hepatobiliary disorders | 6 | (1.9%) | 5 | (1.6%) |
| HLT: Hepatocellular damage and hepatitis NEC | 6 | (1.9%) | 4 | (1.3%) |
| Hepatic steatosis | 6 | (1.9%) | 4 | (1.3%) |
| SKIN AND SUBCUTANEOUS TISSUE DISORDERS | 38 | (11.9%) | 29 | (9.2%) |
| HLGT: Angioedema and urticaria | 4 | (1.3%) | 3 | (0.9%) |
| HLT: Urticarias | 4 | (1.3%) | 2 | (0.6%) |
| Urticaria | 4 | (1.3%) | 2 | (0.6%) |
| HLGT: Epidermal and dermal conditions | 25 | (7.9%) | 15 | (4.7%) |
| HLT: Dermatitis and eczema | 8 | (2.5%) | 7 | (2.2%) |
| Eczema | 2 | (0.6%) | 4 | (1.3%) |
| HLT: Pruritus NEC | 8 | (2.5%) | 3 | (0.9%) |
| Pruritus | 6 | (1.9%) | 3 | (0.9%) |
| HLGT: Skin appendage conditions | 9 | (2.8%) | 11 | (3.5%) |
| HLT: Apocrine and eccrine gland disorders | 7 | (2.2%) | 7 | (2.2%) |
| Hyperhidrosis | 6 | (1.9%) | 7 | (2.2%) |
| MUSCULOSKELETAL AND CONNECTIVE TISSUE | 68 | (21.4%) | 60 | (19.0%) |
| DISORDERS | | | | |
| HLGT: Joint disorders | 29 | (9.1%) | 22 | (7.0%) |
| HLT: Joint related signs and symptoms | 22 | (6.9%) | 14 | (4.4%) |
| Arthralgia | 19 | (6.0%) | 13 | (4.1%) |
| HLT: Osteoarthropathies | 2 | (0.6%) | 7 | (2.2%) |
| Osteoarthritis | 2 | (0.6%) | 7 | (2.2%) |
| HLGT: Muscle disorders | 10 | (3.1%) | 11 | (3.5%) |
| HLT: Muscle pains | 3 | (0.9%) | 5 | (1.6%) |
| Myalgia | 2 | (0.6%) | 5 | (1.6%) |
| Any class | 257 | (80.8%) | 264 | (83.5%) |
| HLT: Muscle related signs and symptoms NEC | 6 | (1.9%) | 5 | (1.6%) |
| Muscle spasms | 5 | (1.6%) | 5 | (1.6%) |
| HLGT: Musculoskeletal and connective tissue disorders NEC | 31 | (9.7%) | 31 | (9.8%) |
| HLT: Musculoskeletal and connective tissue pain and | 30 | (9.4%) | 30 | (9.5%) |
| discomfort | | | | |
| Back pain | 19 | (6.0%) | 16 | (5.1%) |
| Musculoskeletal pain | 5 | (1.6%) | 5 | (1.6%) |
| Neck pain | 4 | (1.3%) | 5 | (1.6%) |
| Pain in extremity | 8 | (2.5%) | 7 | (2.2%) |
| HLGT: Synovial and bursal disorders | 4 | (1.3%) | 5 | (1.6%) |
| HLT: Bursal disorders | 4 | (1.3%) | 2 | (0.6%) |
| Bursitis | 4 | (1.3%) | 2 | (0.6%) |
| HLGT: Tendon, ligament and cartilage disorders | 3 | (0.9%) | 9 | (2.8%) |
| HLT: Tendon disorders | 1 | (0.3%) | 8 | (2.5%) |
| Tendonitis | 0 | | 7 | (2.2%) |
| GENERAL DISORDERS AND ADMINISTRATION SITE | 69 | (21.7%) | 51 | (16.1%) |
| CONDITIONS | | | | |
| HLGT: Administration site reactions | 26 | (8.2%) | 8 | (2.5%) |
| HLT: Injection site reactions | 25 | (7.9%) | 7 | (2.2%) |
| Injection site erythema | 5 | (1.6%) | 0 | |
| Injection site pain | 5 | (1.6%) | 0 | |
| Injection site pruritus | 5 | (1.6%) | 0 | |
| Injection site reaction | 5 | (1.6%) | 2 | (0.6%) |
| HLGT: Body temperature conditions | 5 | (1.6%) | 2 | (0.6%) |
| HLT: Febrile disorders | 5 | (1.6%) | 2 | (0.6%) |
| Pyrexia | 5 | (1.6%) | 2 | (0.6%) |
| Any class | 257 | (80.8%) | 264 | (83.5%) |
| HLGT: General system disorders NEC | 46 | (14.5%) | 44 | (13.9%) |
| HLT: Asthenic conditions | 26 | (8.2%) | 24 | (7.6%) |
| Asthenia | 9 | (2.8%) | 10 | (3.2%) |
| Fatigue | 16 | (5.0%) | 9 | (2.8%) |
| Malaise | 2 | (0.6%) | 7 | (2.2%) |
| HLT: Oedema NEC | 8 | (2.5%) | 9 | (2.8%) |
| Oedema peripheral | 6 | (1.9%) | 6 | (1.9%) |
| HLT: Pain and discomfort NEC | 13 | (4.1%) | 8 | (2.5%) |
| Chest pain | 4 | (1.3%) | 2 | (0.6%) |
| Discomfort | 4 | (1.3%) | 0 | |
| INVESTIGATIONS | 36 | (11.3%) | 45 | (14.2%) |
| HLGT: Endocrine investigations (incl sex hormones) | 6 | (1.9%) | 5 | (1.6%) |
| HLT: Gastrointestinal, pancreatic and APUD hormone | 5 | (1.6%) | 5 | (1.6%) |
| analyses | | | | |
| Blood calcitonin increased | 5 | (1.6%) | 5 | (1.6%) |
| HLGT: Gastrointestinal investigations | 13 | (4.1%) | 17 | (5.4%) |
| HLT: Digestive enzymes | 12 | (3.8%) | 14 | (4.4%) |
| Lipase increased | 10 | (3.1%) | 12 | (3.8%) |
| HLGT: Physical examination topics | 5 | (1.6%) | 4 | (1.3%) |
| HLT: Physical examination procedures | 5 | (1.6%) | 4 | (1.3%) |
| Weight decreased | 5 | (1.6%) | 2 | (0.6%) |
| INJURY, POISONING AND PROCEDURAL COMPLICATIONS | 32 | (10.1%) | 26 | (8.2%) |
| HLGT: Bone and joint injuries | 11 | (3.5%) | 8 | (2.5%) |
| HLT: Limb injuries NEC (incl traumatic amputation) | 7 | (2.2%) | 6 | (1.9%) |
| Joint sprain | 4 | (1.3%) | 3 | (0.9%) |
| Any class | 257 | (80.8%) | 264 | (83.5%) |
| HLGT: Injuries NEC | 23 | (7.2%) | 13 | (4.1%) |
| HLT: Muscle, tendon and ligament injuries | 10 | (3.1%) | 4 | (1.3%) |
| Epicondylitis | 5 | (1.6%) | 0 | |
| HLT: Non-site specific injuries NEC | 8 | (2.5%) | 7 | (2.2%) |
| Fall | 4 | (1.3%) | 2 | (0.6%) |

| | | | |
|---|---|---|---|
| HLGT: Medication errors | 0 | 5 | (1.6%) |
| HLT: Maladministrations | 0 | 4 | (1.3%) |
| Expired drug administered | 0 | 4 | (1.3%) |

TEAE: Treatment emergent adverse event,
SOC: System organ class,
HLGT: High level group term,
HLT: High level term,
PT: Preferred term
MedDRA version: 13.1
Note:
Table sorted by SOC internationally agreed order and HLGT, HLT, PT by alphabetic order.
Only SOC with at least one PT ≧1% in at least one group are presented.

1. A method for the prevention of hypoglycaemia in diabetes mellitus type 2 comprising administering
(a) desPro³⁶Exendin-4(1-39)-Lys₆-NH₂ or/and a pharmaceutically acceptable salt thereof, and
(b) metformin or/and a pharmaceutically acceptable salt thereof,
to a subject in need thereof.

2. The method of claim 1, wherein desPro³⁶Exendin-4(1-39)-Lys₆-NH₂ or/and a pharmaceutically acceptable salt thereof is administered subcutaneously.

3. The method of claim 1, wherein the metformin is administered orally.

4. The method of claim 1, wherein desPro³⁶Exendin-4(1-39)-Lys₆-NH₂ or/and a pharmaceutically acceptable salt is administered in an add-on therapy to administration of metformin.

5. The method of claim 1, wherein the subject to be treated is obese.

6. The method of claim 5, wherein the subject has a body mass index of at least 30.

7. The method of claim 1, wherein the subject to be treated is an adult subject.

8. The method of claim 1, wherein diabetes mellitus type 2 is not adequately controlled with metformin alone.

9. The method of claim 8, wherein treatment with a dose of at least 1.5 g/day metformin alone for three months does not adequately control diabetes mellitus type 2.

10. The method of claim 1, wherein the subject to be treated has a HbA1c value in the range of 7% to 10%.

11. The method of claim 1, wherein the hypoglycaemia is associated with a plasma glucose concentration of below 60 mg/dL.

12. The method of claim 1, wherein the hypoglycaemia is a symptomatic hypoglycaemia.

13. The method of claim 12, wherein the symptomatic hypoglycaemia is associated with at least one symptom selected from sweating, palpitations, hunger, restlessness, anxiety, fatigue, irritability, headache, loss of concentration, somnolence, psychiatric disorders, visual disorders, transient sensory defects, transient motor defects, confusion, convulsions, and coma.

14. The method of claim 1, wherein the hypoglycaemia is a severe symptomatic hypoglycaemia.

15. The method of claim 14, wherein the hypoglycaemia is associated with a plasma glucose concentration below 36 mg/dL.

16. The method of claim 14, wherein the severe symptomatic hypoglycaemia is associated with acute neurological impairment.

17. The method of claim 16, wherein the acute neurological impairment is at least one selected from somnolence, psychiatric disorders, visual disorders, transient sensory defects, transient motor defects, confusion, convulsions, and coma.

18. A pharmaceutical combination comprising
(a) desPro³⁶Exendin-4(1-39)-Lys₆-NH₂ or/and a pharmaceutically acceptable salt thereof, and
(b) metformin or/and a pharmaceutically acceptable salt thereof,
for the use in the prevention of hypoglycaemia in diabetes mellitus type 2 patients.

19. (canceled)

20. The method of claim 11 wherein the hypoglycaemia is associated with a plasma glucose concentration of below 50 mg/dL.

21. The method of claim 11 wherein the hypoglycaemia is associated with a plasma glucose concentration of below 40 mg/dL.

22. The method of claim 11 wherein the hypoglycaemia is associated with a plasma glucose concentration of below 36 mg/dL.
